(19) United States
(12) Patent Application Publication
(10) Pub. No.: US 2022/0071594 A1
(43) Pub. Date: Mar. 10, 2022

(54) TWO DIMENSIONAL TRANSDUCER ARRAYS FOR ULTRASOUND IMAGING

(71) Applicant: UNIVERSITY OF SOUTHERN CALIFORNIA, Los Angeles, CA (US)

(72) Inventors: Jesse Tong-Pin Yen, La Crescenta, CA (US); Robert G. Wodnicki, Los Angeles, CA (US)

(21) Appl. No.: 17/466,634

(22) Filed: Sep. 3, 2021

Related U.S. Application Data
(60) Provisional application No. 63/074,931, filed on Sep. 4, 2020.

(51) Int. Cl. A61B 8/00; A61B 8/08

(52) U.S. Cl. CPC A61B 8/4494; A61B 8/483; A61B 8/4488

(57) ABSTRACT

Two-dimensional transducers arrays for ultrasound imaging is disclosed. The two-dimensional arrays are suitable for formation of two-dimensional (2D) and/or three-dimensional (3D) ultrasound images. The two-dimensional arrays are suitable for real-time 2D and/or 3D ultrasound imaging. The bowtie transducer arrays and the rectangular transducer arrays are suitable for real-time 2D and/or 3D ultrasound imaging.

### CROSS-REFERENCE TO RELATED APPLICATIONS

This patent application is a non-provisional patent application of, and claims the benefit of, co-pending U.S. Provisional Patent Application No. 63/074,931, that was filed on Sep. 4, 2020, and the entire disclosure of which is hereby incorporated by reference herein.

### BACKGROUND OF THE INVENTION

### Field of the Invention

This invention relates to two dimensional transducers arrays for ultrasound imaging. This invention also relates to two dimensional arrays suitable for formation of two dimensional (2D) and/or three dimensional (3D) ultrasound images. This invention further relates to two dimensional arrays suitable for real-time 2D and/or 3D ultrasound imaging. This invention further relates to bowtie transducer arrays and rectangular transducer arrays suitable for real-time 2D and/or 3D ultrasound imaging.

### Description of the Related Art

Ultrasound probes utilizing 2D array transducers enable reliable real-time 3D imaging. Since their development nearly 30 years ago, rapid advancements in sparse array design and subsequent fully-sampled 2D arrays have occurred. Fully-sampled 2D array transducers are most commonly used in obstetrics, and cardiology, with increasing application in radiology.

The majority of fully-sampled arrays operate in the 2-7 MHz range which is frequently used in obstetrics and cardiology. Recently, Philips® has developed the XL14-3 array for imaging of superficial targets in humans such as the carotid artery with high spatial resolution and excellent contrast to noise ratio. These arrays have a footprint on the order of 1-4 cm² and have tens of thousands of elements enabling both fine focus in azimuth as well as excellent slice thickness uniformity due to electronic focusing in elevation [1].

Startup companies such as Butterfly Networks and Exo Imaging use capacitive micromachined ultrasonic transducers (cMUTs) and piezoelectric micromachined ultrasonic transducers (pMUTs) for imaging systems capable of imaging a wide range of targets. These systems use ASICs to miniaturize and provide electronics in a small form factor. These ASICs are placed in close proximity to the 2D array to simplify cabling and maximize signal-to-noise ratio. ASICs funnel signals from thousands of elements to a more manageable number of system receive channels [1].

Within these ASICs, sub-aperture or micro-beamforming is performed on clusters of elements. Significant academic and industrial research has investigated ASIC designs in which all transmit and receive circuitry fit within the area of an ultrasound element [1-5]. This may lead to compromises in array performance such as lowering transmit voltages and using unipolar pulsers. It may also frequently require relaxing the noise performance of receive amplifiers to greatly reduce the dissipated current in the probe handle.

The main challenge with this architecture is the fact that the transmit and receive circuitry for a particular element may have to sit immediately behind the transducer and may have therefore to occupy a limited area dictated by the 2D array element pitch. This may require the ASIC designer to scale down often complex circuitry to a simplified form with a small number of transistor devices that can fit in the limited available design area.

The problem may especially be acute when both low voltage (LV) and high voltage (HV) devices are used because HV devices may typically be 10× larger than equivalent functionality LV devices due to the requirements for voltage standoff for isolation. The compromise may require the HV circuitry to be reduced in functionality and/or transmit voltage, while the LV circuitry may require sacrifice in noise performance due to the limitation on the size of the input pair which may determine the thermal and 1/f noise of the receive circuit. In addition, HV fabrication processes typically lag LV in process node integration which may mean that the digital circuitry will not be optimized and will also consume significant area behind the elements, further compromising performance. Substrate crosstalk between transmit and receive circuits may also present a challenge with highly integrated systems.

Point-of-care ultrasound is playing an increasingly important role for patient management in the intensive care unit (ICU). In the early 1990s, transthoracic echocardiography was shown to improve patient outcomes with cardiac injury. In the ICU, the most common indications and applications of TTE are the hypotensive patient, patients with chest pain, cardiac trauma, cardiac arrest, mechanically ventilated patients, and post-cardiac surgery patients. Today, system miniaturization, innovations in transducers, and development of training programs have led to increasing use of ultrasound to expedite evaluation, diagnosis, and treatment at the bedside.

3D ultrasound imaging strives to overcome many of the limitations of 2D imaging. Specifically, the diagnostician no longer must mentally integrate a series of 2D images into a 3D interpretation of the anatomy. With 2D imaging, it is not feasible to fine the same image plane on a consistent basis over time as is needed for serial monitoring. 3D ultrasound has greater accuracy and less variability when performing volume measurements. Assumptions normally made to calculate volumes from 2D images no longer must be made with 3D images. 3D imaging is more reproducible than 2D ultrasound make it more suitable for monitoring purposes.

3D ultrasound systems may have different levels of complexity ranging from the use of spatial locators integrated with 2D imaging systems to fully-sampled 2D arrays with true 3D beamforming and steering. The first clinical RT3D scanners employed sparse array technology which allowed for simpler transducer fabrication and system development. A disadvantage of such systems may be the increased sidelobe levels and clutter in the image. Developing fully-sampled 2D arrays having several thousands of elements may technologically be difficult and expensive. Application-specific integrated circuits (ASICs) in the probe handle may funnel the signals from thousands of elements into 64-192 signals for further beamforming in the system. ASICs may also have several disadvantages including a noise-power tradeoff, heat dissipation, and sacrifices in hardware performance.

In recent years, many researchers have explored the use of row-column or top-orthogonal-to-bottom (TOBE) arrays as a way of simplifying transducer design. However, due to the elongated elements of a row-column array, the row-column array may be amenable to steering and focusing at large angles.

### Related Art References

The following documents are related art for the background of this disclosure. One digit or two digit numbers in the box brackets before each reference, correspond to the numbers in the box brackets, and that may be referenced in other parts of this disclosure.
[1] B. Savord and R. Solomon, “Fully sampled matrix transducer for real time 3D ultrasonic imaging,” 2003 IEEE Symposium on Ultrasonics, Honolulu, Hi., USA, 2003, pp. 945-953, Vol. 1.
[2] D.-.D. Liu, D. Brueske, T. Willsie and C. Daft, “Sigma-delta dynamic receive beamforming,” 2008 IEEE Ultrasonics Symposium, Beijing, 2008, pp. 1270-1273.
[3] Karaman M et al., Minimally redundant 2-D array designs for 3-D medical ultrasound imaging, IEEE Trans. Med. Imaging, 28(7), pp. 1056-1061 (2009).
[4] Chen et al., A pitch-matched front-end ASIC with integrated subarray beamforming ASDC for miniature 3D ultrasound probes, IEEE J Solid State Circuits 53(11), pp. 3050-3064 (2018).
[5] Wildes et al., 4D ICE: A 2D array transducer with integrated ASIC in a 10 Fr catheter for real-time 3D intracardiac echocardiography, IEEE Trans. Ultras. Ferro. And Freq. Control 63(12), pp. 2159-2173 (2016).
[6] Wodnicki R et al., Tiled large element 1.75D aperture with dual array modules by adjacent integration of PIN-PMN-PT transducers and custom high voltage switching ASICS, 2019 IEEE Ultrasonics Symposium, Glasgow, United Kingdom, pp. 1955-1958.
[7] Walker k-space.
[8] Jensen JA, A multi-threaded version of Field II, 2014 IEEE Symposium on Ultrasonics, pp. 2229-2232.
[9] Wodnicki R et al, Co-integrated PIN-PMN-PT 2-D array and transceiver electronics by direct assembly using a 3-D printed interposer grid frame, 2020 IEEE Trans. Ultras. Ferro. And Freq. Control, 67(2), pp. 387-401.

The contents of each of these documents are incorporated herein in their entirety by reference.

### SUMMARY OF THE INVENTION

This invention relates to an ultrasound imaging system. This invention relates to two dimensional transducers arrays for ultrasound imaging. This invention also relates to two dimensional arrays suitable for formation of two-dimensional (2D) and/or three-dimensional (3D) ultrasound images. This invention further relates to two dimensional arrays suitable for real-time 2D and/or 3D ultrasound imaging. This invention further relates to bowtie transducer arrays and rectangular transducer arrays suitable for real-time 2D and/or 3D ultrasound imaging.

In one example, the ultrasound imaging system may comprise at least one two-dimensional (2D) transducer array.

In one example, the ultrasound imaging system may be configured to form at least one two-dimensional (2D) ultrasound image of a target and/or at least one three-dimensional (3D) ultrasound image of a target.

In one example, the ultrasound imaging system may be configured to form at least one two-dimensional (2D) ultrasound image of a target in real time and/or at least one three-dimensional (3D) ultrasound image of a target in real-time.

In one example, the at least one 2D transducer array may comprise at least two transducer elements. Each transducer element may be configured to operate in only transmitter mode or in only receiver mode.

In one example, the at least one 2D transducer array may comprise at least one bowtie transducer array.

In one example, the at least one 2D transducer array may comprise two bowtie transducer arrays arranged to be orthogonal to each other.

In one example, the at least one 2D transducer array may comprise two bowtie transducer arrays arranged to be orthogonal to each other. Each bowtie transducer array may comprise at least two transducer elements. The at least two transducer elements of the one of the bowtie transducer arrays may be configured to operate in only transmitter mode. The at least two transducer elements of the other bowtie transducer array may be configured to operate in only receiver mode.

In one example, the at least one 2D transducer array may comprise two bowtie transducer arrays arranged to be orthogonal to each other. Each bowtie transducer array may comprise at least two transducer elements. The at least two transducer elements of the one of the bowtie transducer arrays may be configured to operate in only the transmitter mode. The at least two transducer elements of the other bowtie transducer array may be configured to operate in only receiver mode. Apexes of the at least two bowtie transducer arrays may meet in the center of the 2D transducer array.

In one example, the 2D ultrasound transducer array may comprise at least three transducer elements. The at least one transducer element of the at least three transducer elements may not be sampled.

The ultrasound imaging system of any of the preceding claims or the following claims, wherein the ultrasound imaging system is further configured to apply an inverse filtering to achieve an ultrasound image quality closer to a fully-sampled array.

The ultrasound imaging system of any of the preceding claims or the following claims, wherein the ultrasound imaging system may be a point-of-care real-time three-dimensional ultrasound system that may comprise a sparse transducer array, a beamforming system, and/or a signal processing system of RF data.

In one example, the transducer elements configured to operate only in the transmitter mode and/or the transducer elements configured to operate only in the transmitter mode may be bundled to one system channel.

In one example, the transducer elements may be configured such that fine delays in the transmitter mode and/or the receiver mode combined with coarser delays at a system level to provide beamforming equivalent to an ultrasound imaging system that may comprise a conventional delay-and-sum beamforming system.

In one example, the ultrasound imaging system may be configured to perform an inverse filtering operation in a frequency domain to compensate for a low magnitude response near zero frequency.

In one example, the ultrasound imaging system may comprise a bowtie transducer array. The ultrasound imaging system may be configured to perform an inverse filtering operation in the frequency domain to compensate for a low magnitude response near zero frequency.

In one example, the ultrasound imaging system may be configured to apply an inverse filter. In this example, compensation magnitudes may be limited to a maximum value varying in the range of 5 to 10 to minimize noise when the magnitude of A_{TR,BT} goes to zero, where A_{TR,BT} is the normalized k-space response of a bowtie array.

In one example, the ultrasound imaging system may be configured such that inverse filters may be formed by taking the 3D Fourier Transform of the RF volume data to from a single point target located on axis. The inverse filtering operation may be applied in the frequency domain by first performing a 3D Fourier Transform on the RF volume data from the transducer array. Multiplying the result with the inverse filter, and finally applying an inverse 3D Fourier Transform to this result may form a filtered 3D RF dataset.

In one example, the ultrasound imaging system may be configured such that inverse filters may be formed by taking the 3D Fourier Transform of the RF volume data to form a single point target located on axis. The inverse filtering operation may be applied in the frequency domain by first performing a 3D Fourier Transform on the RF volume data from the transducer array. Multiplying the result with the inverse filter, then applying an inverse 3D Fourier Transform to this result may form a filtered 3D RF dataset. Applying envelope detection and logarithmic compression to the 3D RF dataset may then applied.

In one example, the 2D transducer array may be a rectangular boundary array (RBA). The RBA transducer array may comprise a two parallel transducer arrays operating in only transmitter mode focusing the elevation direction. The RBA transducer array may further comprise a two parallel transducer arrays operating in only receiver mode focusing azimuth direction.

In one example, the 2D transducer array may be a rectangular boundary array (RBA). The RBA transducer array may comprise an N×N 2D array. Said array may comprise only 4N-4 elements. Half of said arrays may be configured to operate in only transmitter mode. The other half may be configured to operate in only receiver mode.

In one example, the ultrasound imaging system may be configured to apply a Barker code to increase a low signal-to-noise ratio (SNR).

In one example, the ultrasound imaging system may be configured to decode a Barker code by applying a mismatched filter.

In one example, the ultrasound imaging system may be configured to apply apodization to reduce effects of a high sidelobe level and/or a clutter level of a point spread function.

In one example, the RBA may have more than one rows and/or columns of transducer elements.

In one example, the RBA may have more than one rows and/or columns of transducer elements. Each additional array rows and/or columns may have additional redundancy to improve uniformity.

In one example, the RBA may be arranged as a stairstep like pattern.

In one example, each transducer element of the RBA may have a rectangular front face and may have its own defocusing lens.

In one example, the RBA may have a defocusing lens. The defocusing lens may be a convex defocusing lens.

In one example, the RBA may have a defocusing lens. The defocusing lens may be a concave defocusing lens.

In one example, the RBA may have a defocusing lens. The defocusing lens may have a wedge such that ultrasound energy is steered more toward a center of the array and image, or such that ultrasound energy is steered away from the center of the array.

In one example, all of the transducer elements of the transducer array may be configured to be operated in only transmitter mode or in only receiver mode. In another example, one or more of the transducer elements of the transducer array may be configured to be operated in only transmitter mode and one or more of the transducer elements of the transducer array may be configured to be operated in only receiver mode.

In one example, the ultrasound imaging system may comprise a bowtie array. The transducer elements may be operated in only transmitter mode, using ASICs which may be manufactured using a high voltage ASIC fabrication process with capability greater than above 5V. The transducer elements operated in only the receiver mode may be interfaced using ASICs manufactured using a low voltage ASIC fabrication process with capability less than about 5V.

In one example, the ultrasound imaging system may comprise a bowtie array. The transducer elements operated in only the receiver mode may be implemented using a low voltage multiplexer.

In one example, the ultrasound imaging system may comprise a bowtie array. The transducer elements may be operated in only the receiver mode, which may be implemented using a micro beam-former.

Any combination of above embodiments are within the scope of this disclosure.

Various aspects of the present disclosure are also addressed by the following paragraphs and in the noted combinations:
1. An ultrasound imaging system comprising at least one two-dimensional (2D) transducer array.
2. The ultrasound imaging system of any of the preceding paragraphs or the following paragraphs, wherein the ultrasound imaging system is configured to form at least one two-dimensional (2D) ultrasound image of a target or at least one three-dimensional (3D) ultrasound image of a target.
3. The ultrasound imaging system of any of the preceding paragraphs or the following paragraphs, wherein the ultrasound imaging system is configured to form at least one two-dimensional (2D) ultrasound image of a target in real time or at least one three-dimensional (3D) ultrasound image of a target.in real-time.
4. The ultrasound imaging system of any of the preceding paragraphs or the following paragraphs, wherein the at least one 2D transducer array comprises at least two transducer elements, and wherein each transducer element is configured to operate in only transmitter mode or in only receiver mode.
5. The ultrasound imaging system of any of the preceding paragraphs or the following paragraphs, wherein the at least one 2D transducer array comprises at least one bowtie transducer array.
6. The ultrasound imaging system of any of the preceding paragraphs or the following paragraphs, wherein the at least one 2D transducer array comprises two bowtie transducer arrays arranged to be orthogonal to each other.
7. The ultrasound imaging system of any of the preceding paragraphs or the following paragraphs, wherein the at least one 2D transducer array comprises two bowtie transducer arrays arranged to be orthogonal to each other, wherein each bowtie transducer array comprises at least two transducer elements, and wherein the at least two transducer elements of the one of the bowtie transducer arrays is configured to operate in only transmitter mode whereas the at least two transducer elements of the other bowtie transducer array is configured to operate in only receiver mode.
8. The ultrasound imaging system of any of the preceding paragraphs or the following paragraphs, wherein the at least one 2D transducer array comprises two bowtie transducer arrays arranged to be orthogonal to each other, wherein each bowtie transducer array comprises at least two transducer elements, wherein the at least two transducer elements of the one of the bowtie transducer arrays is configured to operate in only the transmitter mode whereas the at least two transducer elements of the other bowtie transducer array is configured to operate in only receiver mode, and wherein apexes of the at least two bowtie transducer arrays meet in the center of the 2D transducer array.
9. The ultrasound imaging system of any of the preceding paragraphs or the following paragraphs, wherein the 2D ultrasound transducer array comprises at least three transducer elements, and wherein at least one transducer element of the at least three transducer elements is not sampled.
10. The ultrasound imaging system of any of the preceding paragraphs or the following paragraphs, wherein the ultrasound imaging system is further configured to apply an inverse filtering to achieve an ultrasound image quality closer to a fully-sampled array.
11. The ultrasound imaging system of any of the preceding paragraphs or the following paragraphs, wherein the ultrasound imaging system is a point-of-care real-time three-dimensional ultrasound system that comprises a sparse transducer array, a beamforming system, and/or a signal processing system of RF data.
12. The ultrasound imaging system of any of the preceding paragraphs or the following paragraphs, wherein the transducer elements is configured to operate only in the transmitter mode and/or the transducer elements is configured to operate only in the transmitter mode are bundled to one system channel.
13. The ultrasound imaging system of any of the preceding paragraphs or the following paragraphs, wherein the transducer elements are configured such that fine delays in the transmitter mode and/or the receiver mode combined with coarser delays at a system level to provide beamforming equivalent to an ultrasound imaging system that comprises a conventional delay-and-sum beamforming system.
14. The ultrasound imaging system of any of the preceding paragraphs or the following paragraphs, wherein the ultrasound imaging system is configured to perform an inverse filtering operation in a frequency domain to compensate for a low magnitude response near zero frequency.
15. The ultrasound imaging system of any of the preceding paragraphs or the following paragraphs, wherein the ultrasound imaging system comprises a bowtie transducer array, wherein the ultrasound imaging system is configured to perform an inverse filtering operation in the frequency domain to compensate for a low magnitude response near zero frequency.
16. The ultrasound imaging system of any of the preceding paragraphs or the following paragraphs, wherein the ultrasound imaging system is configured to apply an inverse filter, wherein compensation magnitudes are limited to a maximum value varying in the range of 5 to 10 to minimize noise when the magnitude of A_{TR,BT} goes to zero, where A_{TR,BT} is the normalized k-space response of a bowtie array.
17. The ultrasound imaging system of any of the preceding paragraphs or the following paragraphs, wherein the ultrasound imaging system is configured such that inverse filters are formed by taking the 3D Fourier Transform of the RF volume data to from a single point target located on axis, then the inverse filtering operation is applied in the frequency domain by first performing a 3D Fourier Transform on the RF volume data from the transducer array, and then multiplying the result with the inverse filter and finally applying an inverse 3D Fourier Transform to this result forms a filtered 3D RF dataset.
18. The ultrasound imaging system of any of the preceding paragraphs or the following paragraphs, wherein the ultrasound imaging system is configured such that inverse filters are formed by taking the 3D Fourier Transform of the RF volume data to form a single point target located on axis, then the inverse filtering operation is applied in the frequency domain by first performing a 3D Fourier Transform on the RF volume data from the transducer array, and then multiplying the result with the inverse filter and then applying an inverse 3D Fourier Transform to this result forms a filtered 3D RF dataset, and finally applying envelope detection and logarithmic compression to the 3D RF dataset.
19. The ultrasound imaging system of any of the preceding paragraphs or the following paragraphs, wherein the 2D transducer array is a rectangular boundary array (RBA), wherein the RBA transducer array comprises a two parallel transducer arrays operating in only transmitter mode focusing the elevation direction, and wherein the RBA transducer array further comprises a two parallel transducer arrays operating in only receiver mode focusing the azimuth direction.
20. The ultrasound imaging system of any of the preceding paragraphs or the following paragraphs, wherein the 2D transducer array is a rectangular boundary array (RBA), wherein the RBA transducer array comprises an N×N 2D array, wherein said array comprises only 4N-4 elements, and wherein half of said arrays are configured to operate in only transmitter mode and the other half of said arrays are configured to operate in only receiver mode.
21. The ultrasound imaging system of any of the preceding paragraphs or the following paragraphs, wherein the ultrasound imaging system is configured to apply a Barker code to increase a low signal-to-noise ratio (SNR).
22. The ultrasound imaging system of any of the preceding paragraphs or the following paragraphs, wherein the ultrasound imaging system is configured to decode a Barker code by applying a mismatched filter.
23. The ultrasound imaging system of any of the preceding paragraphs or the following paragraphs, wherein the ultrasound imaging system is configured to apply apodization to reduce the effects of a high sidelobe level and/or a clutter level of a point spread function.
24. The ultrasound imaging system of any of the preceding paragraphs or the following paragraphs, wherein the RBA has more than one rows and/or columns of transducer elements.
25. The ultrasound imaging system of any of the preceding paragraphs or the following paragraphs, wherein the RBA has more than one rows and/or columns of transducer elements, wherein each additional array rows and/or columns has additional redundancy to improve uniformity.
26. The ultrasound imaging system of any of the preceding paragraphs or the following paragraphs, wherein the RBA is arranged as a stairstep like pattern.
27. The ultrasound imaging system of any of the preceding paragraphs or the following paragraphs, wherein each transducer element of the RBA may have a rectangular front face and may have its own defocusing lens.
28. The ultrasound imaging system of any of the preceding paragraphs or the following paragraphs, wherein the RBA has a defocusing lens, and wherein the defocusing lens is a convex defocusing lens.
29. The ultrasound imaging system of any of the preceding paragraphs or the following paragraphs, wherein the RBA has a defocusing lens, and wherein the defocusing lens is a concave defocusing lens.
30. The ultrasound imaging system of any of the preceding paragraphs or the following paragraphs, wherein the RBA has a defocusing lens, and wherein the defocusing lens has a wedge such that ultrasound energy is steered more toward a center of the array and image or such that ultrasound energy is steered away from the center of the array.
31. The ultrasound imaging system of any of the preceding paragraphs or the following paragraphs, wherein each of the transducer elements of the transducer array is configured to be operated in only transmitter mode or in only receiver mode, wherein the transducer array includes at least one transducer element configured to operate in only transmitter mode and includes at least one transducer element configured to operate in only receiver mode.
32. The ultrasound imaging system of any of the preceding paragraphs or the following paragraphs, wherein the ultrasound imaging system comprises a bowtie array, wherein the elements operated in only transmitter mode are interfaced directly to transmit electronics which are manufactured using a high voltage ASIC fabrication process with capability greater than above 5V, and wherein the transducer elements operated in only the receiver mode are interfaced directly to transmit electronics which are implemented using a low voltage ASIC fabrication process with capability less than about 5V.
33. The ultrasound imaging system of any of the preceding paragraphs or the following paragraphs, wherein the ultrasound imaging system comprises a bowtie array, and wherein the transducer elements operated in only the receiver mode are interfaced directly to receive electronics which are implemented using a low voltage multiplexer.
34. The ultrasound imaging system of any of the preceding paragraphs or the following paragraphs, wherein the ultrasound imaging system comprises a bowtie array, and wherein the transducer elements operated in only the receiver mode are interfaced directly to transmit electronics which are implemented using a micro beam-former fabricated using a low voltage ASIC process.
35. An ultrasound imaging system comprising an ultrasound transducer array, said transducer array comprising:

a plurality of elements disposed in a plurality of columns and in a plurality of rows;

a first group of said plurality of elements disposed in opposing relation to a second group of said plurality of elements;

wherein a number of elements in each column of each of said first group and said second group is reduced proceeding in a direction of a central portion of said transducer array;

wherein each of said first group and said second group comprises a first side, a second side, and a third side, wherein said second and third sides of each of said first group and said second group extend from opposite ends of a corresponding said first side and converge toward one another proceeding in a direction of said central portion of said transducer array, wherein said first side of said first group is disposed oppositely of said first side of said second group; and

wherein each said element of each of said first group and said second group is a configured to operate in a first common mode, wherein said first common mode is only one of transmit or receive.
36. The ultrasound imaging system of paragraph 35, wherein all said elements of each of said first group and said second group include only transmit elements.
37. The ultrasound imaging system of paragraph 35, wherein all said elements of each of said first group and said second group include only receive elements.
38. The ultrasound imaging system of any of paragraphs 35-37, wherein said second side and said third side of said first group converge to a single first element, and wherein said second side and said third side of said second group converge to a single second element.
39. The ultrasound imaging system of paragraph 38, wherein said first element of said first group is disposed in an adjacent column and in an adjacent row to said second element of said second group.
40. The ultrasound imaging system of any of paragraphs 35-39, wherein a first end element of said first side of said first group is disposed in an adjacent row to a first end element of said first side of said second group.
41. The ultrasound imaging system of paragraph 40, wherein a second end element of said first side of said first group is disposed in an adjacent row to a second end element of said first side of said second group.
42. The ultrasound imaging system of any of paragraphs 35-41, wherein a first perimeter side of said transducer array comprises said first side of said first group, and wherein a second perimeter side of said transducer array that is opposite said first perimeter side comprises said first side of said second group.
43. The ultrasound imaging system of any of paragraphs 35-42, wherein said first group and said second group collectively define a bowtie arrangement.
44. The ultrasound imaging system of paragraph 35, wherein said transducer array further comprises:

a third group of said plurality of elements disposed in opposing relation to a fourth group of said plurality of elements;

wherein a number of elements in each row of each of said third group and said fourth group is reduced proceeding in a direction of said central portion of said transducer array;

wherein each of said third group and said fourth group comprises a first side, a second side, and a third side, wherein said second and third sides of each of said third group and said fourth group extend from opposite ends of a corresponding said first side and converge toward one another proceeding in a direction of said central portion of said transducer array, wherein said first side of said third group is disposed oppositely of said first side of said fourth group.
45. The ultrasound imaging system of paragraph 44, wherein each said element of each of said third group and said fourth group is a configured to operate in a second common mode, wherein said second common mode is only the other of transmit or receive.
46. The ultrasound imaging system of any of paragraphs 44-45, wherein said second side and said third side of each of said first group, said second group, said third group, and said fourth group converge to a different single element.
47. The ultrasound imaging system of paragraph 46:

wherein said single element of said first group is disposed in an adjacent column and in an adjacent row to said single element of said second group; and

wherein said single element of said third group is disposed in an adjacent column and in an adjacent row to said single element of said fourth group.
48. The ultrasound imaging system of paragraph 47, wherein a center of said transducer array is defined by adjoining portions of said single element of each of said first group, said second group, said third group, and said fourth group.
49. The ultrasound imaging system of any of paragraphs 44-48, wherein said first group and said second group collectively define a first bowtie arrangement and said third group and said fourth group collectively define a second bowtie arrangement.
50. The ultrasound imaging system of paragraph 49, wherein said first bowtie arrangement and said second bowtie arrangement are at least generally orthogonal to one another.
51. The ultrasound imaging system of any of paragraphs 44-50, wherein said first group, said second group, said third group, and said fourth group are operable for 3D imaging.
52. The ultrasound imaging system of any of paragraphs 35-51, wherein there is a common number of said elements in each said row and each said column of said transducer array.
53. The ultrasound imaging system of any of paragraphs 35-52, wherein each said element of said transducer array is at least one of the same size, has a square-shaped front surface through which a corresponding transmit or receive function is provided, and is individually controllable.
54. An ultrasound imaging system comprising an ultrasound transducer array, said transducer array comprising a plurality of elements disposed in a plurality of columns and in a plurality of rows, said transducer array comprising:

a first group of multiple said elements, wherein said first group includes at least two adjacent columns, and wherein a first side of said transducer array comprises said first group;

a second group of multiple said elements, wherein said second group includes at least two adjacent columns, and wherein a second side of said transducer array that is opposite said first side comprises said second group;

a third group of multiple said elements, wherein said third group includes at least two adjacent rows, and wherein a third side of said transducer array comprises said third group;

a fourth group of multiple said elements, wherein said fourth group includes at least two adjacent rows, and wherein a fourth side of said transducer array that is opposite said third side comprises said fourth group;

wherein said third side and said fourth side each extend between said first side and said second side;

wherein each said element of each of said first group and said second group is a configured to operate in a first common mode, wherein said first common mode is only one of transmit or receive; and

wherein each said element of each of said third group and said fourth group is a configured to operate in a second common mode, wherein said second common mode is only the other of transmit or receive.
55. The ultrasound imaging system of paragraph 54, wherein each of said first group, said second group, said third group, and said fourth group is rectangular.
56. The ultrasound imaging system of any of paragraphs 54-55, wherein each of said first group, said second group, said third group, and said fourth group each include a common number of said elements and are of a common size.
57. The ultrasound imaging system of paragraph 54:

wherein a number of said elements in a first column of said first group is greater than a number of said elements in an adjacent column of said first group, wherein said first column of said first group is on a perimeter of said transducer array;

wherein a number of said elements in a second column of said second group is greater than a number of said elements in an adjacent column of said second group, wherein said second column of said second group is on said perimeter of said transducer array;

wherein a number of said elements in a first row of said third group is greater than a number of said elements in an adjacent row of said third group, wherein said first row of said third group is on said perimeter of said transducer array; and

wherein a number of said elements in a second row of said fourth group is greater than a number of said elements in an adjacent row of said fourth group, wherein said second row of said fourth group is on said perimeter of said transducer array.
58. The ultrasound imaging system of paragraph 57:

wherein there are two more said elements in said first column of said first group than said adjacent column of said first group;

wherein there are two more said elements in said second column of said second group than said adjacent column of said second group;

wherein there are two more said elements in said first row of said third group than said adjacent row of said third group; and

wherein there are two more said elements in said second row of said fourth group than said adjacent row of said fourth group.
59. The ultrasound imaging system of any of paragraphs 54-58, wherein said first group, said second group, said third group, and said fourth group are operable for 3D imaging.
60. The ultrasound imaging system of any of paragraphs 54-58, wherein said transducer array further comprises a plurality of rectangular elements.
61. The ultrasound imaging system of paragraph 60, wherein said plurality of rectangular elements are of a common size.
62. The ultrasound imaging system of any of paragraphs 60-61, wherein each said rectangular element is larger in a first dimension than each said element of each of said first group, said second group, said third group, and said fourth group in the same said first dimension.
63. The ultrasound imaging system of any of paragraphs 60-62, wherein said first group, said second group, said third group, and said fourth group are collectively disposed about said plurality of rectangular elements.
64. The ultrasound imaging system of any of paragraphs 60-63, wherein a center of said transducer array corresponds with a center of said plurality of rectangular elements.
65. The ultrasound imaging system of any of paragraphs 60-64, wherein said first group, said second group, said third group, and said fourth group are operable for 3D imaging, and wherein said plurality of rectangular elements are operable for 2D imaging.
66. The ultrasound imaging system of any of paragraphs 54-65, wherein each said element of said transducer array is at least one of the same size, has a square-shaped front surface through which a corresponding transmit or receive function is provided, and individually controllable.
67. An ultrasound imaging system comprising an ultrasound transducer array, said transducer array comprising a plurality of elements having a rectangular front surface and that are of a common size, said transducer array comprising:

a first group of a plurality of said elements, wherein a first side of said transducer array comprises said first group;

a second group of a plurality of said elements, wherein a second side of said transducer array that is opposite said first side comprises said second group, wherein said elements are disposed in a common first orientation for each of said first group and said second group, and wherein said first group and said second group include a common number of said elements;

a third group of a plurality of said elements, wherein a third side of said transducer array comprises said third group;

a fourth group of a plurality of said elements, wherein a fourth side of said transducer array that is opposite said third side comprises said fourth group, wherein said elements are in a common second orientation for each of said third group and said fourth group, wherein said third group and said fourth group include a common number of said elements, and wherein said first orientation is different from said second orientation;

wherein said third side and said fourth side each extend between said first side and said second side;

wherein each said element of each of said first group and said second group is a configured to operate in a first common mode, wherein said first common mode is only one of transmit or receive; and

wherein each said element of each of said third group and said fourth group is a configured to operate in a second common mode, wherein said second common mode is only the other of transmit or receive.
68. The ultrasound imaging system of paragraph 67, wherein said first group, said second group, said third group, and said fourth group each include a common number of said elements.
69. The ultrasound imaging system of any of paragraphs 67-68, wherein each said element of said transducer array is individually controllable.
70. The ultrasound imaging system of any of paragraphs 67-69, wherein each element for said transducer array is either in said first group, said second group, said third group, or said fourth group.
71. The ultrasound imaging system of any of paragraphs 67-70, wherein said first orientation is at least substantially orthogonal to said second orientation.
72. The ultrasound imaging system of any of paragraphs 67-71, wherein a width dimension is a largest dimension of each said element for its corresponding said front surface, wherein said width dimension extends from a corresponding side of said transducer array in a direction of an opposite side of said transducer array.
73. The ultrasound imaging system of any of paragraphs 67-72, wherein each said element of each of said first group and said second group is a configured to operate only transmit mode.
74. The ultrasound imaging system of any of paragraphs 67-73, wherein each said element of each of said first group, said second group, said third group, and said fourth group comprises a defocusing lens.
75. The ultrasound imaging system of paragraph 74, wherein each said defocusing lens comprises one of a convex or concave exterior.
76. The ultrasound imaging system of any of paragraphs 74-75, further comprising a steering wedge for each said defocusing lens.
77. The ultrasound imaging system of any of paragraphs 67-76, wherein said first group, said second group, said third group, and said fourth group are operable for 3D imaging.
78. The ultrasound imaging system of any of paragraphs 35-77, wherein the transducers are fabricated using a micro-machining process.
79. The ultrasound imaging system of any of paragraphs 35-77, wherein the transducers are fabricated using a composite material.

These, as well as other components, steps, features, objects, benefits, and advantages, will now become clear from a review of the following detailed description of illustrative examples, the accompanying drawings, and the claims.

For purposes of summarizing the disclosure, certain aspects, advantages, and novel feature are discussed herein. It is to be understood that not necessarily all such aspects, advantages, or features will be embodied in any particular embodiment of the disclosure, and an artisan would recognize from the disclosure herein a myriad of combinations of such aspects, advantages, or features.

### BRIEF DESCRIPTION OF THE DRAWINGS

The drawings are of illustrative embodiments. They do not illustrate all embodiments. Other embodiments may be used in addition or instead. Details that may be apparent or unnecessary may be omitted to save space or for more effective illustration. Some embodiments may be practiced with additional components or steps and/or without all of the components or steps that are illustrated. When the same numeral appears in different drawings, it refers to the same or like components or steps. The entire contents of these patent applications are incorporated herein by reference. The patent application file contains these and additional drawings and photos executed in color. Copies of this patent application file with color drawings and photos will be provided by the United States Patent and Trademark Office upon request and payment of the necessary fee.

FIG. 1 illustrates an orthogonal bowtie array layout for an ultrasound transducer array.

FIG. 1A is an exploded view of the ultrasound transducer array of FIG. 1.

FIG. 2 illustrates K-space coverage of the fully-sampled array (top row) and bowtie array (bottom row), with 3D views being shown in the left column and with azimuthal cross-sections being shown in the right column.

FIG. 3 illustrates K-space magnitude of the inverse filter in 3-D view (left) and azimuthal cross-section (right).

FIG. 4 illustrates on-axis beamplots of the A) Fully-sampled array, B) Bowtie array, C) Bowtie array with inverse filtering, and D) Azimuthal cross-sectional view.

FIG. 5 illustrates off-axis beamplots of the A) Fully-sampled array, B) Bowtie array, C) Bowtie array with inverse filtering, and D) Azimuthal cross-sectional view.

FIG. 6 illustrates simulated point target images using the fully-sampled array (top row), bowtie array (center row), and bowtie array with inverse filtering (bottom row), as well as azimuth B-scans (left column), elevational B-scans (middle column), and C-scan (right column).

FIG. 7 illustrates simulated images of 8 mm diameter spherical anechoic cysts using the fully-sampled array (top row), bowtie array (center row), and bowtie array with inverse filtering (bottom row), as well as azimuth B-scans (left column), elevational B-scans (middle column), and C-scan (right column).

FIG. 8 illustrates an exemplary rectangular boundary array.

FIG. 9A illustrates a plot of a 13-bit Barker code.

FIG. 9B illustrates a plot of coefficients of a mismatched filter.

FIG. 9C illustrates decoded and normalized results.

FIG. 10 illustrates simulated point target images using the fully-sampled array (top row), RBA with coded excitation only (center row), and RBA with coded excitation, NLA, and DAX (bottom row), as well as azimuth B-scan (left column), elevation B-scan (middle column), and C-scan at 60 depth (right column).

FIG. 11 illustrates simulated point target images using the fully-sampled array (top row), RBA with coded excitation only (center row), and RBA with coded excitation, NLA, and DAX (bottom row), as well as azimuth B-scan (left column), elevation B-scan (middle column), and C-scan at 60 depth (right column).

FIG. 12 illustrates an example of a 2-row RBA for an ultrasound transducer array.

FIG. 13 illustrates an example of a stairstep ultrasound array.

FIG. 14 illustrates an example of a multi-row RBA.

FIG. 14A in an enlarged plan view of one of the elements of the ultrasound transducer array of FIG. 14.

FIG. 15 is a cross-section of an exemplary element with a defocusing lens.

FIG. 16 shows the defocusing lens of FIG. 15 in combination with a steering wedge.

FIG. 17 is an example of another ultrasound transducer array.

FIG. 18 is a block diagram of an ultrasound imaging system.

### DETAILED DESCRIPTION

Illustrative examples are now described. Other examples may be used in addition or instead. Details that may be apparent or unnecessary may be omitted to save space or for a more effective presentation. Some examples may be practiced with additional components or steps and/or without all of the components or steps that are described.

This disclosure relates to two dimensional transducers arrays for ultrasound imaging. This disclosure also relates to two dimensional arrays suitable for formation of two-dimensional (2D) and/or three-dimensional (3D) ultrasound images. This disclosure further relates to two dimensional arrays suitable for real-time 2D and/or 3D ultrasound imaging. This disclosure further relates to bowtie transducer arrays and rectangular transducer arrays suitable for real-time 2D and/or 3D ultrasound imaging.

In one example, this disclosure relates to a 2D array design to alleviate the ASIC design challenges and compromises disclosed above. This 2D array design may use all available elements but any given element may be used in transmit only or receive only. Both transmit and receive apertures, which have the shape of a bowtie, may be arranged orthogonal to each other. The interface electronics to drive and receive signals for the transmit and receive apertures respectively may be fabricated separately using separate HV and LV ASICs. The apertures may then be tiled together. The performance of the bowtie array may be analyzed through a series of Field II simulations and compared to a fully-sampled array in terms of resolution, sidelobe levels, and contrast. An inverse filtering approach may be applied to achieve image quality closer to the fully-sampled array.

This disclosure also relates to a point-of-care RT3D ultrasound system that comprises sparse array design, beamforming, and/or signal processing of RF data.

In one example, the ultrasound imaging system may comprise at least one two dimensional (2D) transducer array.

In one example, the ultrasound imaging system may be configured to form at least one two dimensional (2D) ultrasound image of a target and/or at least one three dimensional (3D) ultrasound image of a target.

In one example, the ultrasound imaging system may be configured to form at least one two-dimensional (2D) ultrasound image of a target in real time and/or at least one three-dimensional (3D) ultrasound image of a target in real-time.

In one example, the at least one 2D transducer array may comprise at least two transducer elements. Each transducer element may be configured to operate in only transmitter mode or in only receiver mode.

In one example, the at least one 2D transducer array may comprise at least one bowtie transducer array.

In one example, the at least one 2D transducer array may comprise two bowtie transducer arrays arranged to be orthogonal to each other.

In one example, the at least one 2D transducer array may comprise two bowtie transducer arrays arranged to be orthogonal to each other. Each bowtie transducer array may comprise at least two transducer elements. The at least two transducer elements of the one of the bowtie transducer arrays may be configured to operate in only transmitter mode. The at least two transducer elements of the other bowtie transducer array may be configured to operate in only receiver mode.

In one example, the at least one 2D transducer array may comprise two bowtie transducer arrays arranged to be orthogonal to each other. Each bowtie transducer array may comprise at least two transducer elements. The at least two transducer elements of the one of the bowtie transducer arrays may be configured to operate in only the transmitter mode. The at least two transducer elements of the other bowtie transducer array may be configured to operate in only receiver mode. Apexes of the at least two bowtie transducer arrays may meet in the center of the 2D transducer array.

In one example, the 2D ultrasound transducer array may comprise at least three transducer elements. The at least one transducer element of the at least three transducer elements may not be sampled.

In one example, the ultrasound imaging system is further configured to apply an inverse filtering to achieve an ultrasound image quality closer to a fully-sampled array.

In one example, the ultrasound imaging system may be a point-of-care real-time three-dimensional ultrasound system that may comprise a sparse transducer array, a beamforming system, and/or a signal processing system of RF data.

In one example, the transducer elements configured to operate only in the transmitter mode and/or the transducer elements configured to operate only in the transmitter mode may be bundled to one system channel.

In one example, the transducer elements may be configured such that fine delays in the transmitter mode and/or the receiver mode combined with coarser delays at a system level to provide beamforming equivalent to an ultrasound imaging system that may comprise a conventional delay-and-sum beamforming system.

In one example, the ultrasound imaging system may be configured to perform an inverse filtering operation in a frequency domain to compensate for a low magnitude response near zero frequency.

In one example, the ultrasound imaging system may comprise a bowtie transducer array. The ultrasound imaging system may be configured to perform an inverse filtering operation in the frequency domain to compensate for a low magnitude response near zero frequency.

In one example, the ultrasound imaging system may be configured to apply an inverse filter. In this example, compensation magnitudes may be limited to a maximum value varying in the range of 5 to 10 to minimize noise when the magnitude of A_{TR,BT} (the normalized k-space response of the bowtie array) goes to zero.

In one example, the ultrasound imaging system may be configured such that inverse filters may be formed by taking the 3D Fourier Transform of the RF volume data to from a single point target located on axis. The inverse filtering operation may be applied in the frequency domain by first performing a 3D Fourier Transform on the RF volume data from the transducer array. Multiplying the result with the inverse filter, and finally applying an inverse 3D Fourier Transform to this result may form a filtered 3D RF dataset.

In one example, the ultrasound imaging system may be configured such that inverse filters may be formed by taking the 3D Fourier Transform of the RF volume data to from a single point target located on axis. The inverse filtering operation may be applied in the frequency domain by first performing a 3D Fourier Transform on the RF volume data from the transducer array. Multiplying the result with the inverse filter, then applying an inverse 3D Fourier Transform to this result may form a filtered 3D RF dataset. Applying envelope detection and logarithmic compression to the 3D RF dataset may then applied.

In one example, the 2D transducer array may be a rectangular boundary array (RBA). The RBA transducer array may comprise a two parallel transducer arrays operating in only transmitter mode focusing the elevation direction. The RBA transducer array may further comprise a two parallel transducer arrays operating in only receiver mode focusing the azimuth direction.

In one example, the 2D transducer array may be a rectangular boundary array (RBA). The RBA transducer array may comprise an N×N 2D array. Said array may comprise only 4N-4 elements. Half of said arrays may be configured to operate in only transmitter mode. The other half may be configured to operate in only receiver mode.

In one example, the ultrasound imaging system may be configured to apply a Barker or other (e.g., Golay) code to increase a low signal-to-noise ratio (SNR).

In one example, the ultrasound imaging system may be configured to decode a Barker code by applying a mismatched filter.

In one example, the ultrasound imaging system may be configured to apply apodization to reduce effects of a high sidelobe level and/or a clutter level of a point spread function.

In one example, the RBA may have more than one rows and/or columns of transducer elements.

In one example, the RBA may have more than one rows and/or columns of transducer elements. Each additional array rows and/or columns may have additional redundancy to improve uniformity.

In one example, the RBA may be arranged as a stairstep like pattern.

In one example, each transducer element of the RBA may have a rectangular front face and have its own defocusing lens.

In one example, the RBA may have a defocusing lens. The defocusing lens may be a convex defocusing lens.

In one example, the RBA may have a defocusing lens. The defocusing lens may be a concave defocusing lens.

In one example, the RBA may have a defocusing lens. The defocusing lens may have a wedge such that ultrasound energy is steered more toward a center of the array and image or such that ultrasound energy is steered away from the center of the array.

In one example, each of the transducer elements of the transducer array may be configured to be operated in only transmitter mode or in only receiver mode, with the transducer array including at least one transducer element configured to operate in only transmitter mode and including at least one transducer element configured to operate in only receiver mode.

In one example, the ultrasound imaging system may comprise a bowtie array. The transducer elements may be operated in only transmitter mode, the interface electronics for which may be manufactured using a high voltage ASIC fabrication process with capability greater than above 5V. The transducer elements operated in only the receiver mode may be interfaced to interface electronics manufactured using a low voltage ASIC fabrication process with capability less than about 5V.

In one example, the ultrasound imaging system may comprise a bowtie array. The transducer elements operated in only the receiver mode may be interfaced to electronics which are implemented using a low voltage multiplexer.

In one example, the ultrasound imaging system may comprise a bowtie array. The transducer elements may be operated in only the receiver mode, which may be interfaced to electronics which are implemented using a micro beam-former comprising low voltage fabricated ASICs.

Any combination of above embodiments are within the scope of this disclosure.

### Example 1. Bowtie Array Layout

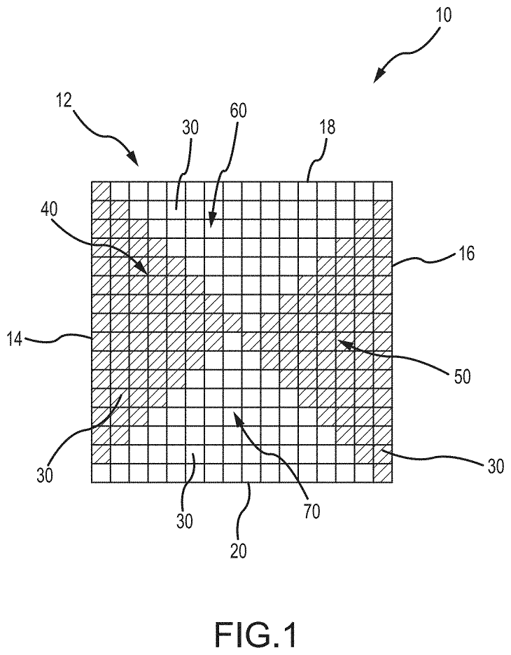

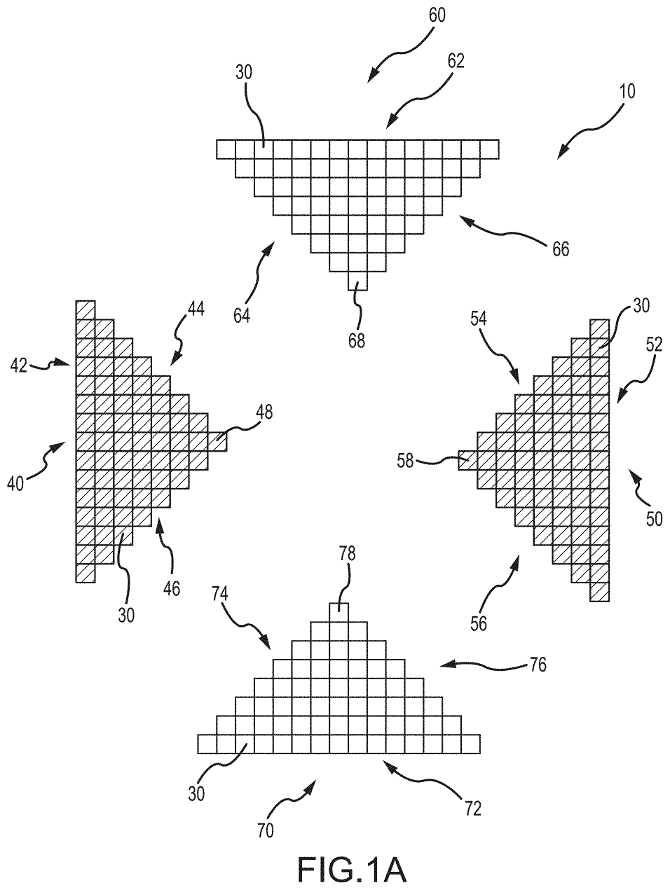

For illustrative purposes, a bowtie array layout of a 16×162D array 10 is shown in FIG. 1. By way of initial summary, the array 10 may be characterized as consisting of four triangles whose apexes meet at least substantially at the center of the array 10. Certain groups of elements are used in transmit only and other groups of elements are used in receive only. These groups of elements are interfaced to separate transmit and receive interface electronics which are implemented using both high voltage and low voltage Application Specific Integrated Circuit (ASIC) fabrication processes. It is assumed that separate transmit and receive ASICs are capable of micro-beamforming where clusters of transmit only elements or receive only elements are bundled to one system channel. At the element level, fine delays in transmit and receive combined with coarser delays at the system level provide beamforming equivalent to conventional delay-and-sum beamforming.

The ultrasound transducer array 10 of FIG. 1 may be characterized as having a perimeter 12. This perimeter 12 may be defined by a first or left side 14, an oppositely disposed second or right side 16, a third or upper side 18, and an oppositely disposed fourth or lower side 20. A plurality of elements 30 are disposed in a plurality of rows and columns for the ultrasound transducer array 10. An entirety of the ultrasound transducer array 10 is defined by a first group 40 and a second group 50 that are disposed in opposing relation to one another and that may be characterized as defining a first bowtie arrangement, along with a third group 60 and a fourth group 70 that are disposed in opposing relation to one another and that may be characterized as defining a second bowtie arrangement. The first bowtie arrangement and the second bowtie arrangement may be characterized as being disposed/oriented at least substantially orthogonal to one another.

Each of the groups 40, 50, 60, and 70, include a plurality of the noted elements 30. Each element 30 of both the first group 40 and the second group 50 may be configured to operate only in a first common mode that is one of transmit or receive, while each element 30 of both the third group 60 and the fourth group 70 may be configured to operate only in a second common mode that is the other of transmit or receive. That is, each of the elements 30 of both the first group 40 and the second group 50 may be configured only to operate as a transmit element, while each of the elements 30 of both the third group 60 and the fourth group 70 may be configured only to operate as a receive element. Alternatively, each of the elements 30 of both the first group 40 and the second group 50 may be configured only to operate as a receive element, while each of the elements 30 of both the third group 60 and the fourth group 70 may be configured only to operate as a transmit element.

Each of the elements 30 in each of the first group 40, second group 50, third group 60, and fourth group 70 may include a front surface through which the corresponding transmit or receive function is provided (this front surface being shown in FIGS. 1 and 1A). This front surface for each of the elements 30 may be in the form of a square and may be of a common size/surface area for each of the elements 30.

With reference to both FIGS. 1 and 1A, the first group 40 may be characterized as being at least generally triangularly-shaped, with a first side 42, a second side 44, and a third side 46. The first or the left side 14 of the ultrasound transducer array 10 may include the first side 42 of the first group 40. The second side 44 and the third side 46 of the first group 40 may converge toward one another proceeding from the first side 42 and in a direction of a central location of the ultrasound transducer array 10, and may converge to a single element 48 of the first group 40.

The second group 50 may be characterized as being at least generally triangularly-shaped, with a first side 52, a second side 54, and a third side 56. The second or the right side 16 of the ultrasound transducer array 10 may include the first side 52 of the second group 50. The second side 54 and the third side 56 of the second group 50 may converge toward one another proceeding from the first side 52 and in a direction of a central location of the ultrasound transducer array 10, and may converge to a single element 58 of the second group 50.

The third group 60 may be characterized as being at least generally triangularly-shaped, with a first side 62, a second side 64, and a third side 66. The third or the upper side 18 of the ultrasound transducer array 10 may include the first side 62 of the third group 60. The second side 64 and the third side 66 of the third group 60 may converge toward one another proceeding from the first side 62 and in a direction of a central location of the ultrasound transducer array 10, and may converge to a single element 68 of the third group 60.

The fourth group 70 may be characterized as being at least generally triangularly-shaped, with a first side 72, a second side 74, and a third side 76. The fourth or the lower side 20 of the ultrasound transducer array 10 may include the first side 72 of the fourth group 70. The second side 74 and the third side 76 of the fourth group 70 may converge toward one another proceeding from the first side 72 and in a direction of a central location of the ultrasound transducer array 10, and may converge to a single element 78 of the fourth group 70. A center of the ultrasound transducer array 10 may correspond with where the elements 48, 58, 68, and 78 meet or adjoin one another.

### Example 2. Imaging System Response in k-Space

The frequency domain or k-space response of an ultrasound imaging system, A_{TR}, may be estimated by the convolution of spatially scaled and reversed representations of the transmit and receive aperture weighting functions indicated by A_{T} and A_{R} respectively:

A_{TR}(fₓ,f_{y})=A_{T}(fₓ^{,}f_{y})*A_{R}(fₓ^{,}f_{y}) Equation (1)

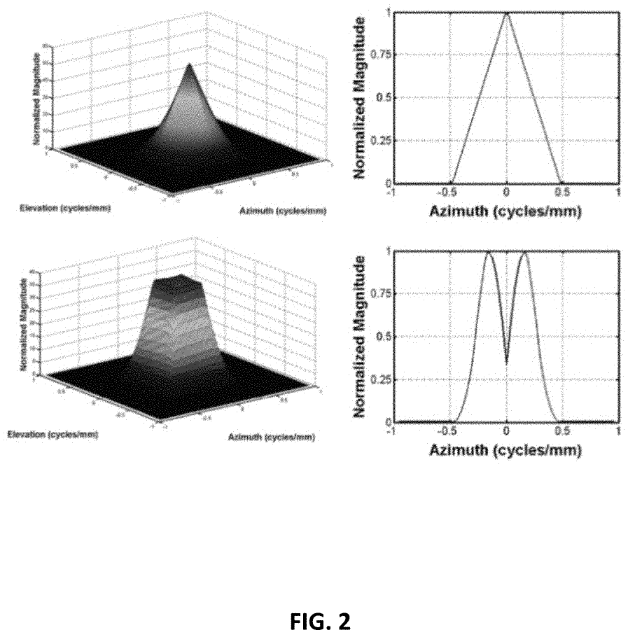

In Equation 1, fₓ and f_{y} are the azimuthal and elevational spatial frequencies respectively, and the asterisk indicates two-dimensional (2-D) convolution. In one example, this may give the results shown in FIG. 2 for the fully sampled array in the top row and the bowtie array in the bottom row. The full 2-D k-space response is shown in the left column, and the azimuthal k-space response of each array is shown in the right column. The elevational k-space response is identical to the azimuthal due to symmetry. As shown, the bowtie array may have the same coverage in k-space as the fully-sampled array. In all figures, the magnitudes have been normalized to themselves. The primary difference is that the bowtie array may have a low magnitude response near zero frequency as indicated by the dimple in the center.

To compensate for this, we perform an inverse filtering operation in the frequency domain. The inverse filter Q(fₓ,f_{y}) is calculated as follows:

$$Q ( {f}_{x} , {f}_{y} ) = \frac{{A}_{TR , FS} ( {f}_{x} , {f}_{y} )}{{A}_{T R , B T} ( {f}_{x} , {f}_{y} )} \qquad Equation ( 2 )$$

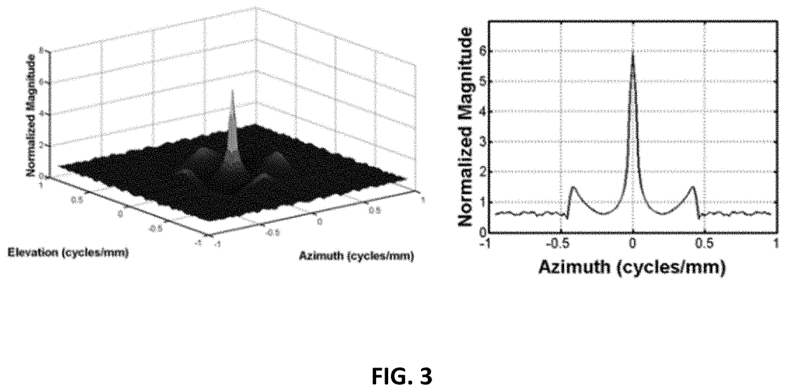

In Equation 2, A_{TR,FS} is the normalized k-space response of a fully-sampled array and A_{TR,BT} is the normalized k-space response of the bowtie array. A limitation of inverse filters may be the tendency to amplify noise when the magnitude of A_{TR,BT} goes to zero. To minimize this in practice, compensation magnitudes may be limited to a maximum value in the range of 5 to 10. FIG. 3 shows the magnitude of an inverse filter where the maximum value is limited to 6. As shown, the largest magnitude is near zero frequency.

### Example 3. Field II Simulations

Simulations of a 64×643 MHz bowtie array and a 64×64 fully-sampled array were carried out using Field II Pro. Using a sound speed of 1540 m/s, the element pitch was equal to about 257 μm, or one-half wavelength. Single point targets located on axis (about 0°, about 0°, about 60 mm) and off-axis (about 40°, about 40°, about 60 mm). Beamwidths at about −6, about −20, and about −40 dB were measured. Simulations involving multiple point targets was also performed. Five point targets were evenly spaced every about 10 degrees from about −20° to about +20°. Additionally, five point targets were spaced evenly in the axial direction from about 40 mm to about 80 mm, giving a total of about 125 point targets. A speckle target with an about 8 mm diameter spherical cyst located at about 60 mm depth was also simulated.

The phantom size was about 50 mm×about 50 mm×about 30 mm. 12 scatterers per resolution volume were used. The transmit and receive focus is set to an about 60 mm distant c-plane centered parallel to the face of the array. A pyramidal volume was scanned having a field of view of about 50°×about 50°×about 80 mm. The line spacing was set to about 0.5°. Therefore, each volume consists of about 101×101 image lines. Beamformed RF data from all image lines was assembled into a 3D volume. Inverse filters were created by taking the 3D Fourier Transform of the RF volume data from a single point target located on axis (about 0°, about 0°, about 60 mm) using both the fully-sampled array and the bowtie array. The inverse filtering operation was then applied in the frequency domain by first performing a 3D Fourier Transform on the RF volume data from the bowtie array and then multiplying the result with the inverse filter. An inverse 3D Fourier Transform was then applied to this result to produce a filtered 3D RF dataset. This 3D RF dataset then underwent envelope detection and logarithmic compression. Images of the point targets are shown in azimuthal and elevation B-scans and C-scans.

### Example 4. Simulated Beamplots and Simulated Images

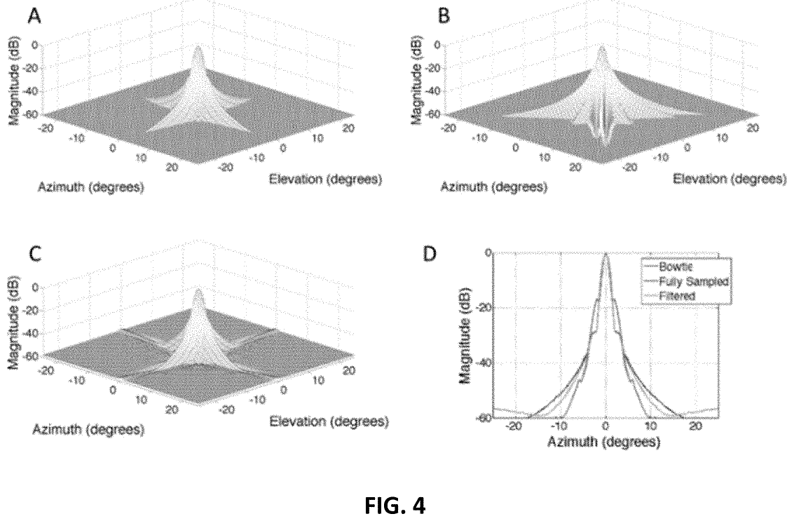

In FIG. 4, on-axis simulated beamplots are from the fully-sampled array (FIG. 4A), the bowtie array alone (FIG. 4B), and the bowtie array with inverse filtering are shown (FIG. 4C). Azimuthal cross-sections for the three scenarios are shown in FIG. 4D. Elevational cross-sections are identical to the azimuth due to symmetry. The −6 dB beamwidths are about 1.75°, about 1.67°, and about 1.75° for the fully-sampled array, the bowtie array, and the bowtie array with inverse filtering. The about −20 dB beamwidths are about 3.25°, about 5.01°, and about 3.25°. The about −40 dB beamwidths are about 10.25°, about 8.5°, and about 9.98°.

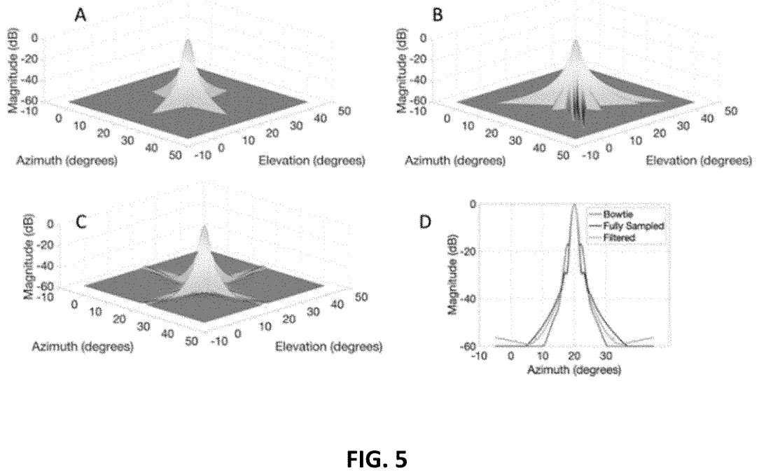

In FIG. 5, off-axis simulated beamplots are from the fully-sampled array (FIG. 5A), the bowtie array alone FIG. 5B), and the bowtie array with inverse filtering are shown (FIG. 5C). Azimuthal cross-sections for the three scenarios are shown in FIG. 5D. Elevational cross-sections are identical to the azimuth due to symmetry. The about −6 dB beamwidths are about 1.88°, about 1.72°, and about 1.90° for the fully-sampled array, the bowtie array, and the bowtie array with inverse filtering, respectively. The about −20 dB beamwidths are about 3.25°, about 5.01°, and about 3.25°. The about −40 dB beamwidths are about 10.25°, about 8.5°, and about 9.98°.

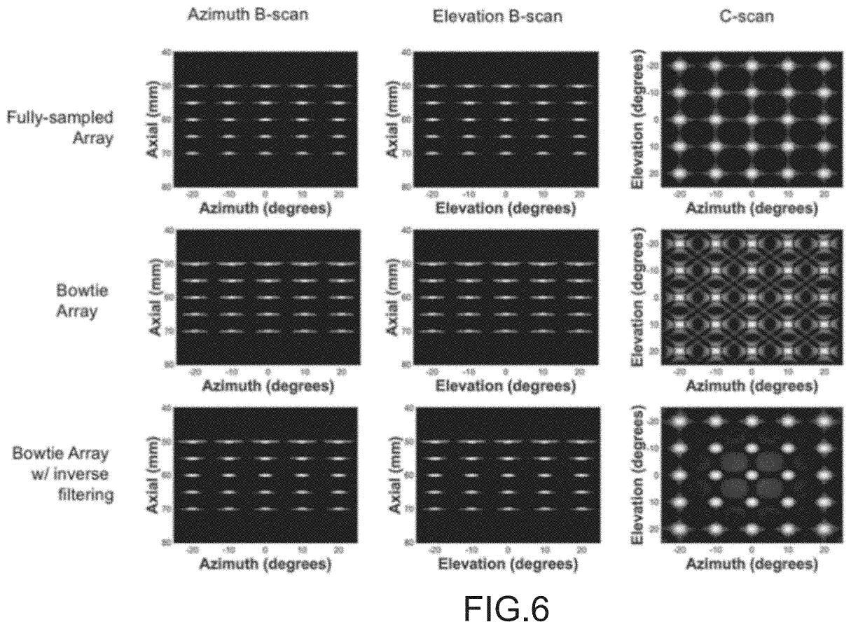

In FIG. 6, simulated images of multiple point targets spaced about every 10° in azimuth and elevation and from about 50 mm to about 70 mm at about 5 mm increments axially are shown. The top row of images are from the fully-sampled array. The middle row is from the bowtie array, and the bottom row is from the bowtie array with inverse filtering using a threshold of about 6. Azimuth and elevation images, shown in the left and middle columns respectively, in all three cases look identical due to symmetry. Without inverse filtering, the bowtie array shows clutter primarily along diagonal directions which is more readily seen in the C-scans shown in the right column. All images are log-compressed and shown on an about 40 dB dynamic range.

With inverse filtering, the bowtie array has clutter levels more comparable to the fully-sampled array. More clutter is seen in the spaces between the points in the range from about −10° to about +10°. Empirically, we observed that the presence and location of the clutter varies with the threshold applied to the inverse filter. Higher thresholds used with inverse filtering displaced the clutter from smaller angles near on-axis to larger lateral angles off-axis.

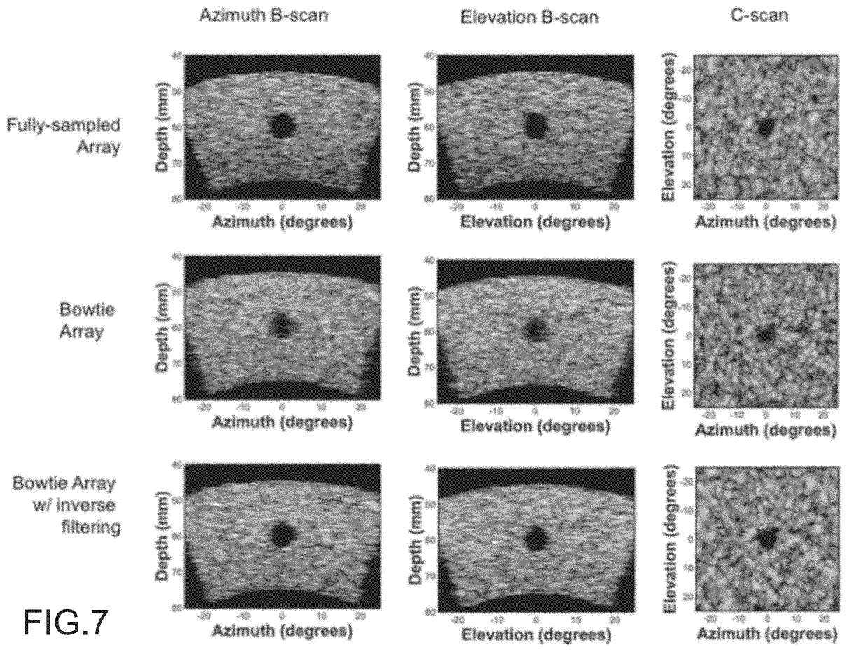

FIG. 7 shows simulated images of an about 8 mm diameter anechoic cyst at about 60 mm depth using the fully-sampled array, bowtie array, and bowtie array with inverse filtering. The CNRs for the fully-sampled array are about 4.45, about 4.36, and about 3.72 for the azimuth B-scan, elevation B-scan and C-scan respectively. For the bowtie array, the CNRs are about 2.67, about 3.02, and about 2.94 for the same respective scans. With inverse filtering applied, the CNRs increase to about 3.73, about 3.80, and about 3.75. Qualitatively, the anechoic cyst with inverse filtering appear more comparable to the images produced by the fully-sampled array. All images are log-compressed and shown on about 40 dB dynamic range.

### Example 5. Orthogonal Bowtie Array

In this example, an orthogonal bowtie array was investigated in which all elements of a 2-D phased array are used either in transmit only or receive only. The performance of the bowtie array was analyzed using a k-space approach and evaluated with computer simulations using Field II pro were used to evaluate spatial resolution and contrast. A modified inverse filter approach was used to compensate for differences between the fully-sampled array and the bowtie array. The differences in the about −6, about −20, and about −40 dB beamwidths between the fully-sampled array and the bowtie array were considered small. However, these differences were most obvious in the anechoic sphere simulation. While the inverse filtering method did improve the CNR, the fully-sampled array still had CNRs that were about 15% to about 20% higher in the azimuthal and elevation B-scans. The C-scan CNRs were considered equivalent. Future simulations will involve evaluating performance with hyperechoic and hypoechoic targets and studying the impact of phase aberration on the bowtie array.

For these arrays, separate transmit and receive ASICs could be designed and optimized individually, enabling the flexibility to select high density low noise processes for the LV receive circuitry, and the best dedicated processes for the HV circuitry. In particular it is important to have access to higher voltage processes (e.g. above 40 Vpp) as these will greatly improve the overall sensitivity of the beamforming process thereby reducing noise and increasing contrast resolution which are critical parameters for expected diagnostic capability in medical imaging ultrasound. These higher voltage processes are inherently less highly integrated than more modern low voltage processes; therefore it is important to be able to separate high voltage and low voltage functionality such that these may be optimized independently to obtain the best possible performance of the overall system.

Array modules including the transducer elements and ASICs would then be assembled together to form the array. In our previous work [6, 9] we have demonstrated highly integrated and tileable transducer and ASIC array modules for constructing large area imaging apertures. For these bowtie arrays, sub-aperture or micro-beamforming processing of the data would still be performed. These array designs achieve comparable image quality to a fully-sampled array but afford the ability to develop separate transmit and receive electronics. This allows, for example the use of BCD HV CMOS processes with fully complementary NMOS and PMOS HV transistors for high quality output drivers with low distortion, while using very high-density processes to implement low noise preamplifiers and sigma-delta converters at every element. The bowtie array design may be useful for applications where size limitations are severe. These applications include intravascular ultrasound, intracardiac echocardiography, transesophageal echocardiography, endoscopic ultrasound, and high frequency (>15 MHz) imaging for ophthalmological applications.

### Example 6. Rectangular Boundary Arrays (RBA)

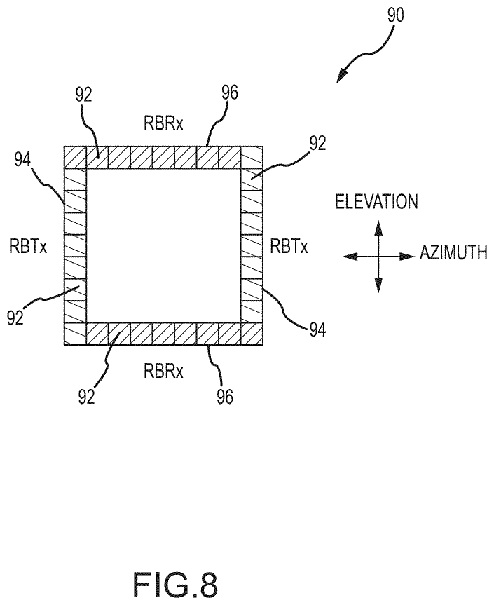

An illustrative example of an 8×8 RBA is shown in FIG. 8 and is identified by reference numeral 90. The RBA 90 includes a pair of oppositely disposed transmit arms 94 (each being defined by a common number of elements 92), along with a pair of oppositely disposed receive arms 96 (each being defined by a common number of elements 92). The transmit arms 94 are orientated perpendicular to the receive arms 96. The RBA 90 may use a two parallel transmit arrays focusing the elevation direction and two parallel receive arrays focusing the azimuth direction. The design of the RBA 90 may drastically simplify 2D array fabrication. For an N×N 2D array, only 4N-4 elements may be needed where half of those may be used in transmit only and the other half may be used in receive only. For example, for a 32×32 array, only 124 of the 1024 elements may be used or about 12.3% of all available elements.

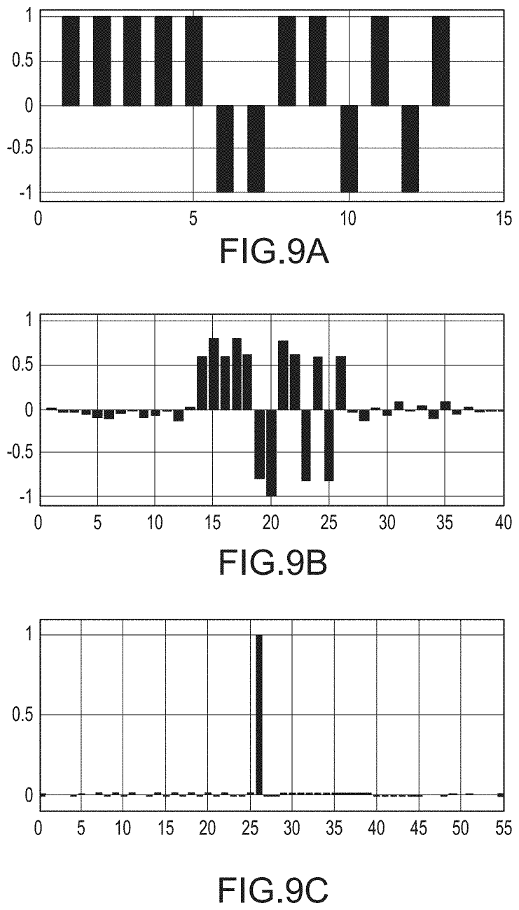

A potential limitation of the RBA 90 may be a low signal-to-noise ratio (SNR) because so few elements are used compare to a fully sampled array. To increase SNR, Barker codes with mismatched filters are used. Barker codes may be used due to their simplicity for implementation because of their biphasic nature. We used the 13-bit Barker code plotted in FIG. 9A. A value of 1 is represented by a 2-cycle pulse having the form sin(2*pi*f0*t) where f0 is the center frequency and a value of −1 is represented by −sin(2*pi*f0*t). Upon receive, the Barker code is decoded with a mismatched filter. The coefficients of the mismatched filter are plotted in FIG. 9B. The decoded and normalized result is shown in FIG. 9C demonstrating a compressed pulse.

### Example 7. Nonlinear Apodization

Due to the high degree of sparseness, the point spread function contains high sidelobe and clutter levels. Signal processing and beamforming strategies are needed to suppress or eliminate clutter signals. A widely used approach is to apply apodization. Conventional apodization applies weighting to signals from individual elements. For example, a Hanning window as the weighting function w(x0)=0.5+0.5*cos(2*pi*xo/D) where x0 is the aperture coordinate and D is the aperture size. Near the focal region, the aperture and the beam have a Fourier Transform relationship. Because of this relationship, a Hanning weighting may also be achieved by convolving the image data with a 5-point kernel of [0.25, 0, 0.5, 0, 0.25] assuming the RF data has been captured with an appropriate line spacing. This 5-point kernel is convolved with the RF data set in for every axial depth and is equivalent to conventional apodization where weightings are applied to individual ultrasound elements. For 3-D imaging a two-dimensional version of the 5-point kernel can be used, and a 2-D dimensional convolution is carried out for each axial depth. This 5-point kernel is expanded to a 5×5 kernel:

$$w [ m , n ] = [ \begin{matrix}0.125 & 0 & 0.25 & 0 & 0.125 \\ 0 & 0.375 & 0 & 0.375 & 0 \\ 0.25 & 0 & 0.5 & 0 & 0.25 \\ 0 & 0.375 & 0 & 0.375 & 0 \\ 0.125 & 0 & 0.25 & 0 & 0.25\end{matrix} ] \qquad ( 1 )$$

In nonlinear apodization approach, data may be apodized uniformly and with a Hanning window using a convolution kernel similar to what is described above. Phase differences and similarities are used to identify and subsequently suppress sidelobe contributions to the signal. In this example, we apply nonlinear apodization to ultrasound signals from an RBA.

As an extension of NLA, we modified version called dual apodization with cross-correlation (DAX). DAX is a technique where received RF echo data is apodized with two different apodization functions. The pair of apodization which showed the highest gains in contrast-to-noise ratio (CNR) experimentally uses complementary square wave apodizations. After apodizing twice, the phase differences in the RF data can then be used to distinguish which echoes are clutter and which echoes are not using normalized cross-correlation at zero lag.

### Example 8. Field II Simulations

Simulations were carried out using Field II Pro. Simulations of a point target phantom where points were evenly spaced in a polar coordinate system. Single point targets located on axis (about 0°, about 0°, about 60 mm) and off-axis (about 40°, about 40°, about 60 mm). Main lobe widths (about −6 dB beamwidth) and integrated sidelobe ratios (ISLR) were calculated. Simulations involving multiple point targets was also performed. Five point targets were evenly spaced every about 10 degrees from about −40° to about +40°. Additionally, five point targets were spaced evenly in the axial direction from about 40 mm to about 80 mm. The transmit and receive focus is set to about 60 mm. A pyramidal volume was scanned having a field of view of about 90°×about 90°×about 100 mm. It was empirically determined that having a line spacing of about 0.5° was optimal. Therefore, each volume consists of about 181×about 181 image lines. Images of the point targets are shown in azimuthal and elevation B-scans as well as C-scans. Images with and without nonlinear apodization and DAX are shown.

### Example 9. Point Target Grid

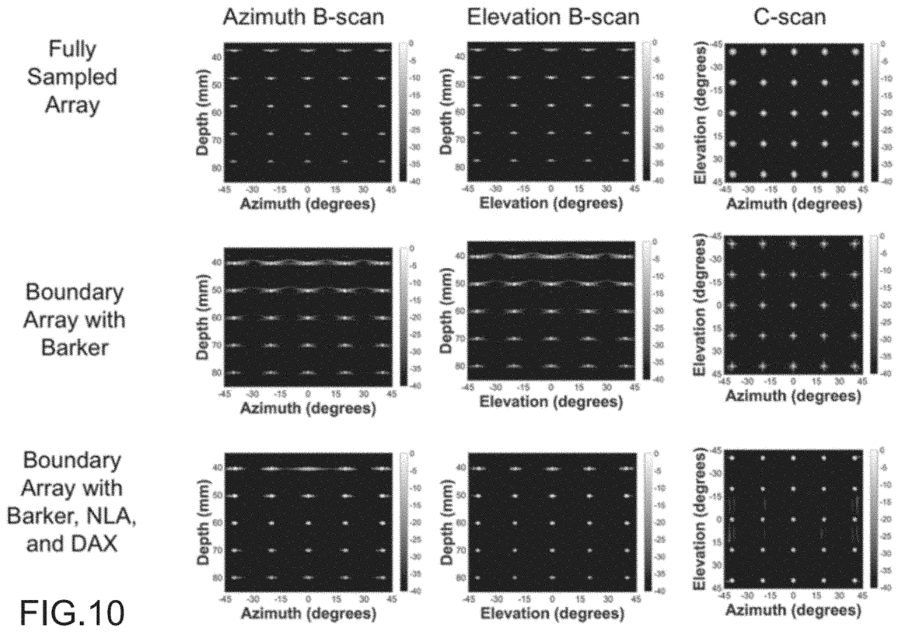

FIG. 10 shows the azimuthal B-scan, elevational B-scan and C-scan at about 60 mm depth of the point target grid using the fully-sampled array, the RBA with coded excitation only, and the RBA with coded excitation, NLA, and DAX. The RBA alone shows higher sidelobe levels that are more prevalent in the B-scans. The fully-sampled array shows slightly larger point targets. This may be due to the fact that the fully-sampled array contains higher magnitudes near zero-frequency than at higher frequency. Whereas the RBA has a more uniform response in its frequency domain representation. The on-axis lateral resolution for the full-sampled array is about 1.73°, about 1.16° for the RBA with no processing, and about 1.38° for the RBA with NLA+DAX. For the off-axis case when the point target is located at (about 40°, about 40°, about 60 mm), the resolutions are about 2.33°, about 1.58°, and about 1.64° for the same respective scenarios.

### Example 10. Speckle Phantom

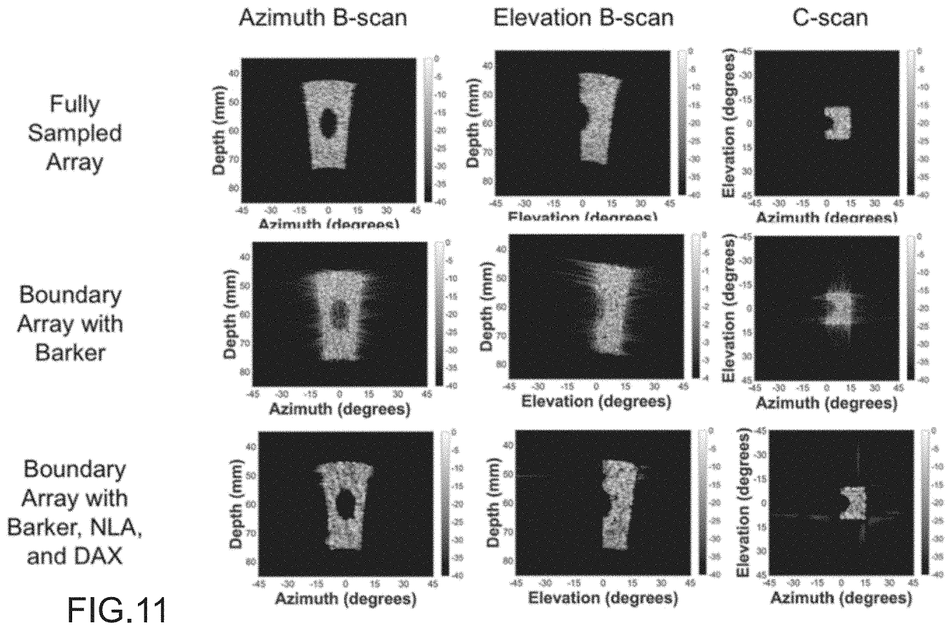

FIG. 11 shows the azimuthal B-scan, elevational B-scan and C-scan at about 60 mm depth using the fully-sampled array, the RBA with coded excitation only, and the RBA with coded excitation, NLA and DAX. The fully-sampled array shows a high contrast anechoic region with well-defined borders in all three scans. The RBA with coded excitation only shows prominent sidelobe and clutter contributions. These unwanted contributions have been suppressed with NLA and DAX. The CNRs for the fully-sampled array, RBA with coded excitation, and RBA with NLA and DAX are about 4.74, about 2.67, and about 6.11 respectively. A moderate improvement in CNR over the fully-sampled is observed although both images appear qualitatively similar.

### Example 11. 3D Ultrasound

3D ultrasound at the point of care may provide a valuable tool to clinicians for serially monitoring disease progression and response to treatment. To minimize the complexity of systems associated with fully-sampled 2D array, we simulated the use of rectangular boundary array combined with clutter suppression techniques. Performance was quantified in terms of spatial resolution and CNR. Comparisons with a fully sampled array were also performed.

### Example 12. Other Examples of RBAs

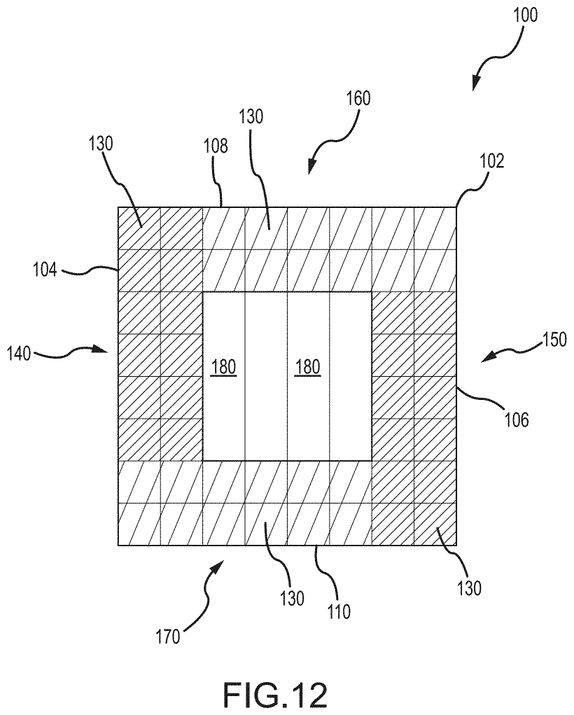

In one example, an RBA may have more rows and columns, as shown in FIG. 12. This may help compensate for variations in element performance (bandwidth, sensitivity, crosstalk, etc.). Each element in the co-array (k-space) may have additional redundancy which will improve uniformity. Redundancy goes up by N2. If N=2, the redundancy goes up by a factor of 4. This may require a denser interconnect but could still potentially avoid the need for an application-specific integrated circuit (ASIC). If an ASIC may be needed, the ASIC design may likely be much simpler than current ASICs for a full-sampled array because fewer connections may be made. An example of a 2-row RBA is shown in FIG. 12.

An ultrasound transducer array is illustrated in FIG. 12, is identified by reference numeral 100, and may be characterized as having a perimeter 102. This perimeter 102 may be defined by a first or left side 104, an oppositely disposed second or right side 106, a third or upper side 108, and an oppositely disposed fourth or lower side 110. The ultrasound transducer array 100 may be characterized as being defined by a first group 140 and a second group 150 that are disposed in opposing relation to one another, along with a third group 160 and a fourth group 170 that are disposed in opposing relation to one another. Each of the groups 140, 150, 160, 170 may be rectangular, may be of a common size, may include a common number of elements 130, or any combination thereof. The array 100 may include any appropriate number of rows and columns of elements 130 (e.g., may be of any appropriate size).

Each element 130 of both the first group 140 and the second group 150 may be configured to operate only in a first common mode that is one of transmit or receive, while each element 130 of both the third group 160 and the fourth group 170 may be configured to operate only in a second common mode that is the other of transmit or receive. That is, each of the elements 130 of both the first group 140 and the second group 150 may be configured only to operate as a transmit element (FIG. 12), while each of the elements 130 of both the third group 160 and the fourth group 170 may be configured only to operate as a receive element (FIG. 12). Alternatively, each of the elements 130 of both the first group 140 and the second group 150 may be configured only to operate as a receive element, while each of the elements 130 of both the third group 160 and the fourth group 170 may be configured only to operate as a transmit element.

Each of the elements 130 in each of the first group 140, second group 150, third group 160, and fourth group 170 may include a front surface through which the corresponding transmit or receive function is provided (this front surface being shown in FIG. 12). This front surface for each of the elements 130 may be in the form of a square and may be of a common size/surface area for each of the elements 130.

The ultrasound transducer array 100 of FIG. 12 further includes a plurality of rectangular elements 180 (four of such elements 180 being illustrated). Each of the rectangular elements 180 may be of a common size. Each rectangular element 180 may be larger in a first dimension (top-to-bottom in FIG. 12) that the same first dimension for the elements 130. The first group 140, second group 150, third group 160, and fourth group 170 are collectively disposed about the rectangular elements 180, with the first group 140, second group 150, third group 160, and fourth group 170 being operable for 3D imaging, and with the rectangular elements 180 being operable for 2D imaging.

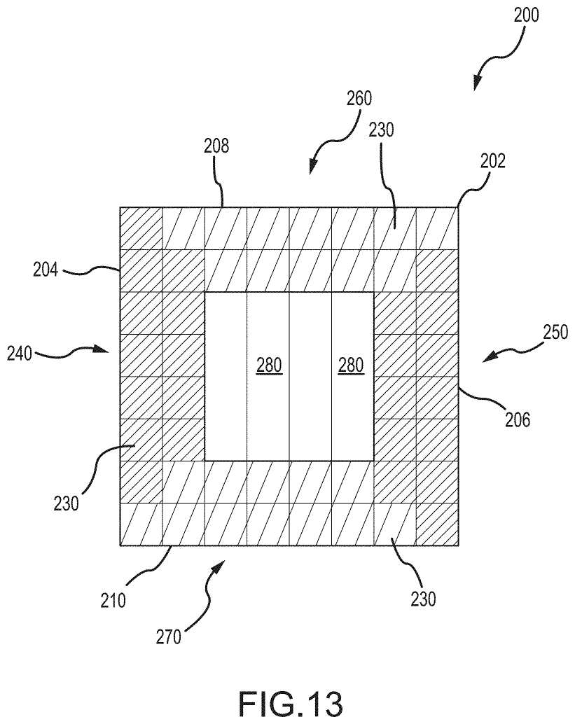

In another example, the transmit/receive arrays may be arranged in a “stairstep”-like pattern and as shown in FIG. 13, instead of rectangular blocks (groups 140, 150, 160, 170) as shown in FIG. 12. An ultrasound transducer array is illustrated in FIG. 13, is identified by reference numeral 200, and may be characterized as having a perimeter 202. This perimeter 202 may be defined by a first or left side 204, an oppositely disposed second or right side 206, a third or upper side 208, and an oppositely disposed fourth or lower side 210. The ultrasound transducer array 200 may be characterized as being defined by a first group 240 and a second group 250 that are disposed in opposing relation to one another, along with a third group 260 and a fourth group 270 that are disposed in opposing relation to one another. The first side 204 of the array 200 includes the first group 240, the second side 206 of the array 200 includes the second group 250, the third side 208 of the array 200 includes the third group 260, and the fourth side 210 of the array 200 includes the fourth group 270.

Each element 230 of both the first group 240 and the second group 250 may be configured to operate only in a first common mode that is one of transmit or receive, while each element 230 of both the third group 260 and the fourth group 270 may be configured to operate only in a second common mode that is the other of transmit or receive. That is, each of the elements 230 of both the first group 240 and the second group 250 may be configured only to operate as a transmit element (FIG. 13), while each of the elements 230 of both the third group 260 and the fourth group 270 may be configured only to operate as a receive element (FIG. 13). Alternatively, each of the elements 230 of both the first group 240 and the second group 250 may be configured only to operate as a receive element, while each of the elements 230 of both the third group 260 and the fourth group 270 may be configured only to operate as a transmit element.

Each of the elements 230 in each of the first group 240, second group 250, third group 260, and fourth group 270 may include a front surface through which the corresponding transmit or receive function is provided (this front surface being shown in FIG. 13). This front surface for each of the elements 230 may be in the form of a square and may be of a common size/surface area for each of the elements 230.

For each of the first group 240 and the second group 250, the number of elements 230 in a given column is reduced proceeding in the direction of a central region of the ultrasound transducer array 200. For instance and as shown in FIG. 13, the outermost column of elements 230 in each of the first group 240 and the second group 250 includes two more elements 230 than the adjacent (and inwardly disposed) column of elements 230. Similarly, for each of the third group 260 and the fourth group 270, the number of elements 230 in a given row is reduced proceeding in the direction of a central region of the ultrasound transducer array 200. For instance and as shown in FIG. 13, the outermost row of elements 230 in each of the third group 260 and the fourth group 270 includes two more elements 230 than the adjacent (and inwardly disposed) row of elements 230.

The ultrasound transducer array 100 of FIG. 13 further includes a plurality of rectangular elements 280 (four of such elements 280 being illustrated—any appropriate number may be utilized). Each of the rectangular elements 280 may be of a common size. Each rectangular element 280 may be larger in a first dimension (top-to-bottom in FIG. 13) than the same first dimension for the elements 230. The first group 240, second group 250, third group 260, and fourth group 270 are collectively disposed about the rectangular elements 180, with the first group 240, second group 250, third group 260, and fourth group 270 being operable for 3D imaging, and with the rectangular elements 280 being operable for 2D imaging.

In both of the two examples of FIGS. 12 and 13, performance may likely increase but at the expense of the need of a denser interconnect, more system channels, and possibly the need for an ASIC.

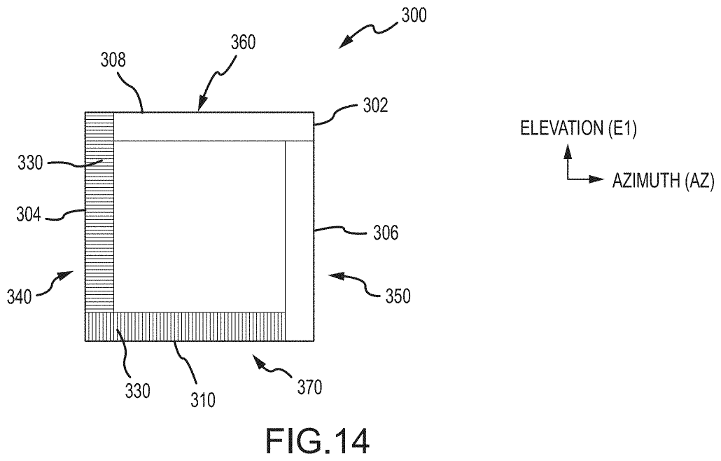

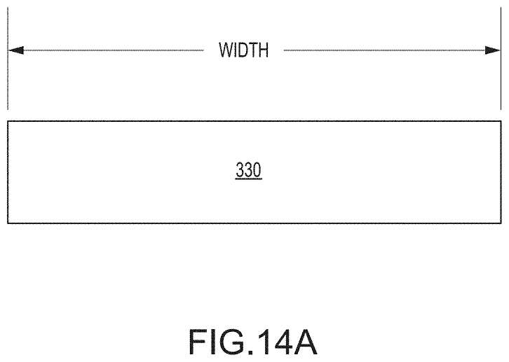

In yet another example, a multi-row boundary array may be built where a defocusing lens on each of the arms of the multi-row RBA may be attached to the front side of the transducer. Each row of the transmit RBA may be treated as one transmit element and each column of the RBA may be treated as one receive element. In FIG. 14, a 64×64 multi-row RBA is shown. Each arm of the RBA may have 56 elements in a stack along the corresponding length dimension of the arm and each such arm may be 8 elements long (or wide). Because each element of the RBA may be larger than a single element of the RBA shown in previous figures. This may alleviate potential electrical impedance issues with having only 1 small element which would have high impedance. The high element source impedance would lead to significant reduction in signal sensitivity when interfaced to a long cable and low impedance system side termination (e.g. 50Ω). Therefore, large element groupings with respectively lower source impedance are advantageous for improved signal sensitivity which in turn improves imaging penetration and contrast to noise ratio. It may be possible to have RBA elements larger than 1×8 elements.

An ultrasound transducer array is illustrated in FIG. 14, is identified by reference numeral 300, and may be characterized as having a perimeter 302. This perimeter 302 may be defined by a first or left side 304, an oppositely disposed second or right side 306, a third or upper side 308, and an oppositely disposed fourth or lower side 310. The ultrasound transducer array 300 may be characterized including a first group 340 and a second group 350 that are disposed in opposing relation to one another, along with a third group 360 and a fourth group 370 that are disposed in opposing relation to one another. The first side 304 of the array 300 includes the first group 340, the second side 306 of the array 300 includes the second group 350, the third side 308 of the array 300 includes the third group 360, and the fourth side 310 of the array 300 includes the fourth group 370.

The first group 340, second group 350, third group 360, and fourth group 370 each include a plurality of elements 330 (the elements 330 not being shown for the second group 350 (similarly configured to the first group 340) of for the third group 360 (similarly configured to the fourth group 370). Each element 330 of both the first group 340 and the second group 350 may be configured to operate only in a first common mode that is one of transmit or receive, while each element 330 of both the third group 360 and the fourth group 370 may be configured to operate only in a second common mode that is the other of transmit or receive. That is, each of the elements 330 of both the first group 340 and the second group 350 may be configured only to operate as a transmit element (FIG. 14), while each of the elements 330 of both the third group 360 and the fourth group 370 may be configured only to operate as a receive element (FIG. 14). Alternatively, each of the elements 330 of both the first group 340 and the second group 350 may be configured only to operate as a receive element, while each of the elements 330 of both the third group 360 and the fourth group 370 may be configured only to operate as a transmit element.

Each of the elements 330 in each of the first group 340, second group 350, third group 360, and fourth group 370 may include a front surface through which the corresponding transmit or receive function is provided (this front surface being shown in each of FIGS. 14 and 14A). This front surface for each of the elements 330 may be rectangular and may be of a common size/surface area for each of the elements 330. The elements 330 of the first group 340 and second group 350 are disposed in a common first orientation, while the elements 330 of the third group 360 and fourth group 370 are disposed in a common second orientation that is different from the first orientation (e.g., the first and second orientations may be orthogonal to each other). The width dimension is the largest dimension of the front surface for each of the elements 330, and this width dimension extends from the side of array 300 along which the corresponding group 340, 350, 360, 370 extends and toward the opposite side of the array 300. For instance, the first group 340 and second group 350 may be a stack of elements 330 (e.g., 56) each having a width (measured in a dimension extending from the first side 304 of the array 300 to the second side 306 of the array 300) that corresponds with a length (measured in a dimension extending from the first side 304 of the array 300 to the second side 306 of the array 300) of multiple elements 330 (e.g., 8) in the stacks for the third group 360 and the fourth group 370. Similarly, the third group 360 and fourth group 370 may be a stack of elements 330 (e.g., 56) each having a width (measured in a dimension extending from the third side 308 of the array 300 to the fourth side 310 of the array 300) that corresponds with a length (measured in a dimension extending from the third side 308 of the array 300 to the fourth side 310 of the array 300) of multiple elements 330 (e.g., 8) in the stacks for the first group 340 and the second group 350.

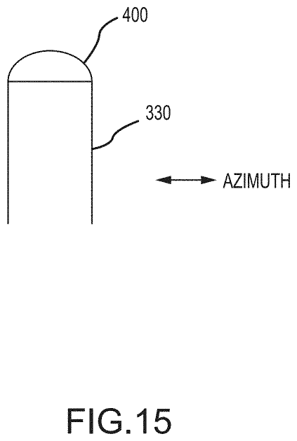

Yet, in another example, a cross-section of an element 300 with a defocusing lens 400 is shown in FIG. 15 (the width dimension for the element 300 being shown in FIG. 15). The convex defocusing lens 400 (convex on an exterior side of the lens 400) may have a sound speed faster than the speed of sound in tissue to create the diverging wavefront. A concave defocusing lens (not shown, but again concave on an exterior side of the lens 400) may also be used if the lens has a sound speed less than the sound speed of tissue. Each element 330 for each of the groups 340, 350, 360, 370 of the array 300 of FIG. 14 may include such a defocusing lens, and including the defocusing lens 400.

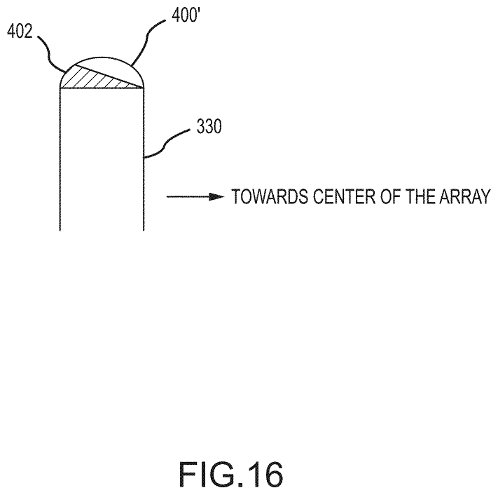

Yet, in another example, the shape/orientation of the defocusing lens 400′ may also be varied as shown in FIG. 16. Since only the edge elements may be used in an RBA, the center of the image may lack sufficient SNR. To alleviate this, the defocusing lens 400′ may be modified by adding a wedge 402 that may steer more of the energy towards the center of the array and image.

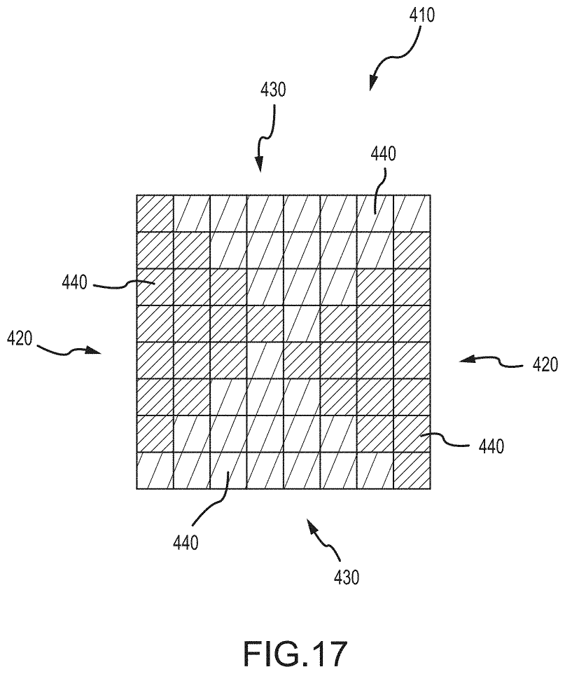

Yet, in another example, a hybrid between a fully sampled array and an RBA, is shown in FIG. 17. By taking the array designs shown in FIG. 12 to the extreme where all of the elements may be used in either transmit or receive, an array 410 consisting of 2 orthogonal “bowties” or bowtie arrangements 420, 430 can be achieved (and thereby similar to the array 10 discussed above in relation to FIG. 1). Shown in FIG. 17, all of the elements 440 may be used, which may either be configured to be used in transmit mode only or in receive mode only. For instance, the bowtie arrangement 420 may have all of its elements 440 configured for only a transmit mode, while the bowtie arrangement 430 may have all of its elements 440 configured for only a receive mode. This array 410 may give a similar beam pattern compared to the other RBA examples disclosed above. The benefits of this design may be: 1) higher signal-to-noise ratio (i.e. improved sensitivity) because more elements are being used, 2) greater degree of co-array redundancy which may lead to lower acoustic clutter, 3) an ASIC may likely be needed for this array design. The ASIC design may be simplified because each element is used in transmit or receive only. No transmit/receive switch may be needed. It may be easier to fit electronics for each element directly underneath the corresponding element due to the reduction in circuit complexity.

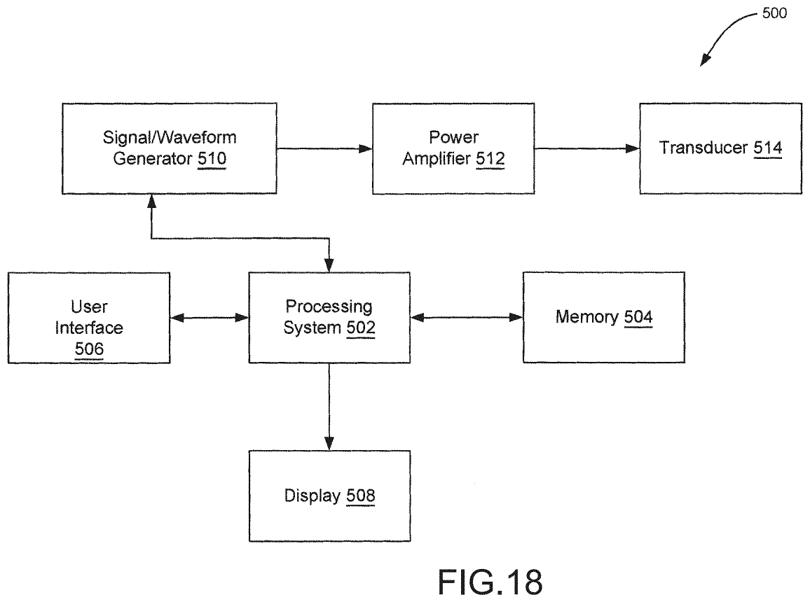

A schematic of an ultrasound system is illustrated in FIG. 18 and is identified by reference numeral 500 (the receive circuitry and accompanying software not being illustrated in FIG. 18). The ultrasound system 500 includes a processing system 502 (e.g., a central processing unit; one or more processors or microprocessors of any appropriate type and utilizing any appropriate processing architecture and including a distributed processing architecture), a signal or waveform generator 510, and an ultrasound transducer 514. The ultrasound transducer 514 may be of any appropriate type and/or configuration, and may be configured to include any of the arrays 10, 100, 200, 300, 410 addressed above. An amplifier or a power amplifier 512 may be disposed between the waveform generator 510 and the ultrasound transducer 514. A user interface 506 of any appropriate type (e.g., a monitor, a keyboard, a mouse, a touchscreen), memory 504, and a display 508 may each be operatively interconnected with the processing system 502. Although the user interface 506, processing system 502, memory 504, and display 508 are illustrated separately from the waveform generator 510, it should be appreciated that one or more of these components (including all of these components) could actually be part of the waveform generator 510.

### Additional Embodiments and Terminology

The components, steps, features, objects, benefits, and advantages that have been discussed are merely illustrative. None of them, nor the discussions relating to them, are intended to limit the scope of protection in any way. Numerous other examples are also contemplated. These include examples that have fewer, additional, and/or different components, steps, features, objects, benefits, and/or advantages. These also include examples in which the components and/or steps are arranged and/or ordered differently.

All of the features disclosed in this specification (including any accompanying exhibits, claims, abstract and drawings), and/or all of the steps of any method or process so disclosed, may be combined in any combination, except combinations where at least some of such features and/or steps are mutually exclusive. The disclosure is not restricted to the details of any foregoing examples. The disclosure extends to any novel one, or any novel combination, of the features disclosed in this specification (including any accompanying claims, abstract and drawings), or to any novel one, or any novel combination, of the steps of any method or process so disclosed.

Those skilled in the art will appreciate that in some examples, the actual steps taken in the processes illustrated or disclosed may differ from those shown in the figures. Depending on the example, certain of the steps described above may be removed, others may be added. For example, the actual steps or order of steps taken in the disclosed processes may differ from those shown in the figure. Depending on the example, certain of the steps described above may be removed, others may be added. For instance, the various components illustrated in the figures may be implemented as software or firmware on a processor, controller, ASIC, FPGA, or dedicated hardware. Hardware components, such as processors, ASICs, FPGAs, and the like, can include logic circuitry. Furthermore, the features and attributes of the specific examples disclosed above may be combined in different ways to form additional examples, all of which fall within the scope of the present disclosure.

Conditional language, such as “can,” “could,” “might,” or “may,” unless specifically stated otherwise, or otherwise understood within the context as used, is generally intended to convey that certain examples include, while other examples do not include, certain features, elements, or steps. Thus, such conditional language is not generally intended to imply that features, elements, or steps are in any way required for one or more examples or that one or more examples necessarily include logic for deciding, with or without user input or prompting, whether these features, elements, or steps are included or are to be performed in any particular example. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Likewise, the term “and/or” in reference to a list of two or more items, covers all of the following interpretations of the word: any one of the items in the list, all of the items in the list, and any combination of the items in the list. Further, the term “each,” as used herein, in addition to having its ordinary meaning, can mean any subset of a set of elements to which the term “each” is applied. Additionally, the words “herein,” “above,” “below,” and words of similar import, when used in this application, refer to this application as a whole and not to any particular portions of this application.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain examples require the presence of at least one of X, at least one of Y, and at least one of Z.

Unless otherwise stated, all measurements, values, ratings, positions, magnitudes, sizes, and other specifications that are set forth in this disclosure are approximate, not exact. They are intended to have a reasonable range that is consistent with the functions to which they relate and with what is customary in the art to which they pertain.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain examples, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

All articles, patents, patent applications, and other publications that have been cited in this disclosure are incorporated herein by reference.

In this disclosure, the indefinite article “a” and phrases “one or more” and “at least one” are synonymous and mean “at least one”.

Relational terms such as “first” and “second” and the like may be used solely to distinguish one entity or action from another, without necessarily requiring or implying any actual relationship or order between them. The terms “comprises,” “comprising,” and any other variation thereof when used in connection with a list of elements in the specification or claims are intended to indicate that the list is not exclusive and that other elements may be included. Similarly, an element preceded by an “a” or an “an” does not, without further constraints, preclude the existence of additional elements of the identical type.

The abstract is provided to help the reader quickly ascertain the nature of the technical disclosure. It is submitted with the understanding that it will not be used to interpret or limit the scope or meaning of the claims. In addition, various features in the foregoing detailed description are grouped together in various examples to streamline the disclosure. This method of disclosure should not be interpreted as requiring claimed examples to require more features than are expressly recited in each claim. Rather, as the following claims reflect, inventive subject matter lies in less than all features of a single disclosed example. Thus, the following claims are hereby incorporated into the detailed description, with each claim standing on its own as separately claimed subject matter.

The various illustrative logical blocks, modules, routines, and algorithm steps described in connection with the embodiments disclosed herein can be implemented as electronic hardware, computer software, or combinations of both. To clearly illustrate this interchangeability of hardware and software, various illustrative components, blocks, modules, and steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

Moreover, the various illustrative logical blocks and modules described in connection with the embodiments disclosed herein can be implemented or performed by a machine, such as a general purpose processor device, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor device can be a microprocessor, but in the alternative, the processor device can be a controller, microcontroller, or state machine, combinations of the same, or the like. A processor device can include electrical circuitry configured to process computer-executable instructions. In another embodiment, a processor device includes an FPGA or other programmable device that performs logic operations without processing computer-executable instructions. A processor device can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor device may also include primarily analog components. For example, some or all of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, routine, or algorithm described in connection with the embodiments disclosed herein can be embodied directly in hardware, in a software module executed by a processor device, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An exemplary storage medium can be coupled to the processor device such that the processor device can read information from, and write information to, the storage medium. In the alternative, the storage medium can be integral to the processor device. The processor device and the storage medium can reside in an ASIC. The ASIC can reside in a user terminal. In the alternative, the processor device and the storage medium can reside as discrete components in a user terminal.

Any combination of above embodiments are within the scope of this disclosure.

What is claimed is:

1. An ultrasound imaging system comprising an ultrasound transducer array, said transducer array comprising:
a plurality of elements disposed in a plurality of columns and in a plurality of rows;
a first group of said plurality of elements disposed in opposing relation to a second group of said plurality of elements;
wherein a number of elements in each column of each of said first group and said second group is reduced proceeding in a direction of a central portion of said transducer array;
wherein each of said first group and said second group comprises a first side, a second side, and a third side, wherein said second and third sides of each of said first group and said second group extend from opposite ends of a corresponding said first side and converge toward one another proceeding in a direction of said central portion of said transducer array, wherein said first side of said first group is disposed oppositely of said first side of said second group; and
wherein each said element of each of said first group and said second group is a configured to operate in a first common mode, wherein said first common mode is only one of transmit or receive.

2. The ultrasound imaging system of claim 1, wherein a first perimeter side of said transducer array comprises said first side of said first group, and wherein a second perimeter side of said transducer array that is opposite said first perimeter side comprises said first side of said second group.

3. The ultrasound imaging system of claim 1, wherein said first group and said second group collectively define a bowtie arrangement.

4. The ultrasound imaging system of claim 1, wherein said transducer array further comprises:
a third group of said plurality of elements disposed in opposing relation to a fourth group of said plurality of elements;
wherein a number of elements in each row of each of said third group and said fourth group is reduced proceeding in a direction of said central portion of said transducer array;
wherein each of said third group and said fourth group comprises a first side, a second side, and a third side, wherein said second and third sides of each of said third group and said fourth group extend from opposite ends of a corresponding said first side and converge toward one another proceeding in a direction of said central portion of said transducer array,
wherein said first side of said third group is disposed oppositely of said first side of said fourth group.

5. The ultrasound imaging system of claim 4, wherein each said element of each of said third group and said fourth group is a configured to operate in a second common mode, wherein said second common mode is only the other of transmit or receive.

6. The ultrasound imaging system of claim 4, wherein said second side and said third side of each of said first group, said second group, said third group, and said fourth group converge to a different single element.

7. The ultrasound imaging system of claim 4, wherein said first group and said second group collectively define a first bowtie arrangement and said third group and said fourth group collectively define a second bowtie arrangement.

8. The ultrasound imaging system of claim 7, wherein said first bowtie arrangement and said second bowtie arrangement are at least generally orthogonal to one another.

9. An ultrasound imaging system comprising an ultrasound transducer array, said transducer array comprising a plurality of elements disposed in a plurality of columns and in a plurality of rows, said transducer array comprising:
a first group of multiple said elements, wherein said first group includes at least two adjacent columns, and wherein a first side of said transducer array comprises said first group;
a second group of multiple said elements, wherein said second group includes at least two adjacent columns, and wherein a second side of said transducer array that is opposite said first side comprises said second group;
a third group of multiple said elements, wherein said third group includes at least two adjacent rows, and wherein a third side of said transducer array comprises said third group;
a fourth group of multiple said elements, wherein said fourth group includes at least two adjacent rows, and wherein a fourth side of said transducer array that is opposite said third side comprises said fourth group;
wherein said third side and said fourth side each extend between said first side and said second side;
wherein each said element of each of said first group and said second group is a configured to operate in a first common mode, wherein said first common mode is only one of transmit or receive; and
wherein each said element of each of said third group and said fourth group is a configured to operate in a second common mode, wherein said second common mode is only the other of transmit or receive.

10. The ultrasound imaging system of claim 9, wherein each of said first group, said second group, said third group, and said fourth group is rectangular.

11. The ultrasound imaging system of claim 9, wherein each of said first group, said second group, said third group, and said fourth group each include a common number of said elements and are of a common size.

12. The ultrasound imaging system of claim 9:
wherein a number of said elements in a first column of said first group is greater than a number of said elements in an adjacent column of said first group, wherein said first column of said first group is on a perimeter of said transducer array;
wherein a number of said elements in a second column of said second group is greater than a number of said elements in an adjacent column of said second group, wherein said second column of said second group is on said perimeter of said transducer array;
wherein a number of said elements in a first row of said third group is greater than a number of said elements in an adjacent row of said third group, wherein said first row of said third group is on said perimeter of said transducer array; and
wherein a number of said elements in a second row of said fourth group is greater than a number of said elements in an adjacent row of said fourth group, wherein said second row of said fourth group is on said perimeter of said transducer array.

13. The ultrasound imaging system of claim 9, wherein said transducer array further comprises a plurality of rectangular elements.

14. The ultrasound imaging system of claim 13, wherein said first group, said second group, said third group, and said fourth group are collectively disposed about said plurality of rectangular elements.

15. The ultrasound imaging system of claim 13, wherein said first group, said second group, said third group, and said fourth group are operable for 3D imaging, and wherein said plurality of rectangular elements are operable for 2D imaging.

16. An ultrasound imaging system comprising an ultrasound transducer array, said transducer array comprising a plurality of elements having a rectangular front surface and that are of a common size, said transducer array comprising:
a first group of a plurality of said elements, wherein a first side of said transducer array comprises said first group;
a second group of a plurality of said elements, wherein a second side of said transducer array that is opposite said first side comprises said second group, wherein said elements are disposed in a common first orientation for each of said first group and said second group, and wherein said first group and said second group include a common number of said elements;
a third group of a plurality of said elements, wherein a third side of said transducer array comprises said third group;
a fourth group of a plurality of said elements, wherein a fourth side of said transducer array that is opposite said third side comprises said fourth group, wherein said elements are in a common second orientation for each of said third group and said fourth group, wherein said third group and said fourth group include a common number of said elements, and wherein said first orientation is different from said second orientation;
wherein said third side and said fourth side each extend between said first side and said second side;
wherein each said element of each of said first group and said second group is a configured to operate in a first common mode, wherein said first common mode is only one of transmit or receive; and
wherein each said element of each of said third group and said fourth group is a configured to operate in a second common mode, wherein said second common mode is only the other of transmit or receive.

17. The ultrasound imaging system of claim 16, wherein said first orientation is at least substantially orthogonal to said second orientation.

18. The ultrasound imaging system of claim 16, wherein a width dimension is a largest dimension of each said element for its corresponding said front surface, wherein said width dimension extends from a corresponding side of said transducer array in a direction of an opposite side of said transducer array.

19. The ultrasound imaging system of claim 16, wherein each said element of each of said first group, said second group, said third group, and said fourth group comprises a defocusing lens.

20. The ultrasound imaging system of claim 19, further comprising a steering wedge for each said defocusing lens.
